(19) United States
(12) Patent Application Publication
(10) Pub. No.: US 2009/0304743 A1
(43) Pub. Date: Dec. 10, 2009

(54) Composition and Methods for Immunisation Using CD1D Ligands

(76) Inventor: Grazia Galli, Siena (IT)

Correspondence Address: NOVARTIS VACCINES AND DIAGNOSTICS INC., INTELLECTUAL PROPERTY- X100B, P.O. BOX 8097, Emeryville, CA, 94662-8097, US

(21) Appl. No.: 12/225,271

(22) PCT Filed: Mar. 15, 2007
(86) PCT No.: PCT/IB2007/001744

§ 371 Date: May 20, 2009

(30) Foreign Application Priority Data
Mar. 15, 2006 (GB) 0605247.6

(51) Int. Cl. A61K 39/145; A61K 39/09; A61K 39/095; A61P 31/12

(52) U.S. Cl. 424/209.1; 424/244.1; 424/250.1

(57) ABSTRACT

The invention relates to immunogenic compositions containing CD1d ligands that induce long-term immunological memory in the absence of booster doses and/or in the absence of multiple priming doses. The invention further relates to immunogenic compositions containing CD1d ligands and antigens from influenza virus, group B streptococcus and serogroup B meningococcus.

### TECHNICAL FIELD

This invention is in the field of vaccine compositions and immunisation methods using vaccine compositions.

### BACKGROUND ART

The first administration of a vaccine composition comprising an antigen from a pathogen induces a primary response against the antigen in the form of both activated cells and memory cells. Subsequent exposure to the antigen (e.g. exposure to the pathogen) induces expansion of memory cells and a secondary response that is faster and greater than the primary response, providing protection against the pathogen.

Although memory cells may persist for months or even years after primary exposure to the antigen, it is generally necessary to provide a booster dose of the antigen to ensure maintenance of long-term immunological memory. Vaccination regimens thus often include several priming injections to provide an initial bank of memory cells and subsequent booster injections at increasing intervals to maintain immunological memory. The requirement for several priming injections and the frequency with which a booster injection is required varies depending on the vaccine and the age of the recipient.

It would be advantageous to be able to reduce the number of priming doses and the frequency and number of booster doses without compromising maintenance of immunological memory. Ideally, it would be preferable to remove the need for additional priming doses and booster doses completely and administer vaccines as a single dose. It is therefore an object of the invention to provide immunogenic compositions that induce long-term immunological memory in the absence of booster doses and/or in the absence of multiple priming doses.

It is a further object of the invention to provide immunogenic compositions comprising antigens from influenza virus, group B streptococcus and serogroup B meningococcus.

### DISCLOSURE OF THE INVENTION

Vaccines often include adjuvants to boost immune activity. Examples of known adjuvants include aluminium salts, oil-in-water emulsions, saponins, cytokines, lipids and CpG oligonucleotides. Currently, only aluminium salts, 3-de-O-acylated monophosphoryl lipid A (‘3dMPL’), and MF59 are approved for human use.

Another molecule known to have adjuvant properties is α-galactosylceramide (α-GalCer or α-GC), a glycolipid, more specifically a glycosylceramide, originally isolated from marine sponges [1]. α-GalCer is a ligand of the MHC class I-like molecule, CD1d, and is presented by CD1d molecules to invariant Natural Killer T (NKT) cells. α-GalCer was originally investigated for its ability to induce a NKT cell response against tumour cells [2]. Invariant NKT cells have been also shown to induce B cell activation, enhancing B cell proliferation and antibody production [3,4]. α-GalCer has been shown to act as an adjuvant for a variety of co-administered protein antigens [5]. Coadministration of α-GalCer with irradiated sporozoites or recombinant viruses expressing a malaria antigen has been shown to enhance the level of protective anti-malaria immunity in mice [6]. α-GalCer has also been shown to act as an adjuvant for a DNA vaccine encoding HIV-1 gag and env genes [7] and to induce a humoral and cellular immune response to influenza virus HA when administered intranasally [8].

Surprisingly, it has now been found that use of a CD1d ligand such as α-GalCer as a vaccine adjuvant not only significantly enhances the antibody response to antigens in the vaccine but also induces an increase in the specific B cell memory pool against those antigens. Specifically, it has been found that administration of a single dose of a composition comprising α-GalCer and an antigen is sufficient to promote an increase in the specific B memory pool that enhances antibody response to challenge with the antigen one year later. The ability of this CD1d ligand to promote an increase in the specific B cell memory pool indicates that use of CD1d ligands as vaccine adjuvants may reduce the number and frequency of priming and boosting doses required to obtain long-term immunological memory.

It has also been found that CD1d ligands are surprisingly effective adjuvants for antigens derived from group B streptococcus, meningococcus serogroup B and for certain influenza virus antigens.

### Methods of Inducing Long-Term Immunological Memory

The invention provides a method of inducing long-term immunological memory to an antigen in a patient in need thereof comprising administering to said patient a composition comprising:
a) said antigen; and
b) a CD1d ligand,
   such that the number and/or frequency of doses of said composition necessary for said patient to be capable of raising an immune response to subsequent exposure to said antigen is reduced compared to administration of said antigen in the absence of a CD1d ligand.

Preferably, the method of the invention reduces the number and/or frequency of doses of said composition necessary for said patient to be capable of raising a protective immune response to subsequent exposure to said antigen compared to administration of said antigen in the absence of a CD1d ligand. By “protective immune response” is meant that the immune response raised to subsequent exposure to the antigen is sufficient to prevent the patient contracting the disease associated with the antigen. Reduction in the number and/or frequency of doses of the composition required to raise a protective immune response to an antigen can be measured by standard methods known in the art.

The method of the invention may reduce the number of doses of a composition comprising an antigen necessary to induce an protective immune response against subsequent exposure to that antigen. Some immunisations currently require three or four priming doses of an antigen to raise a protective immune response to subsequent exposure to an antigen. Preferably, the method of the invention reduces the number of doses required to induce a protective immune response against the antigen to a single priming dose.

Current immunisation methods often also require booster immunisations at increasing intervals to maintain the protective immune response to subsequent exposure to an antigen. For example, immunisations given during infancy typically involve booster doses given months or years after administration of the initial dose. Preferably, the method of the invention reduces the frequency of booster doses of a composition comprising an antigen necessary to maintain a protective immune response against subsequent exposure to the antigen. Preferably the method of the invention allows booster doses to be administered at intervals of more than one year, preferably more than two years, preferably more than 5 years, preferably more than 10 years. According to a preferred embodiment of the invention, the requirement for booster doses is completely eliminated and a single dose of the antigen is sufficient to induce a protective immune response against subsequent exposure to the antigen.

According to one aspect of the invention, there is provided a method of inducing an immune response against an antigen in a patient comprising administering to said patient:
a) said antigen; and
b) a CD1d ligand,
   wherein said antigen and a CD1d ligand were also administered to said patient more than one year previously.

The invention also provides the use of an antigen and a CD1d ligand in the manufacture of a medicament to induce an immune response in a patient, wherein said antigen and a CD1d ligand were also administered to said patient more than 1 year previously.

Preferably the immune response is a protective immune response. Preferably, said antigen and a CD1d ligand were administered to said patient more than 18 months previously, preferably more than 2 years, 5 years, or 10 years previously.

The antigen and CD1d ligand administered to the patient according to this aspect of the invention may be administered as a mixture, i.e. as a single composition comprising both the antigen and CD1d ligand. Alternatively, the antigen and CD1d ligand may be administered sequentially to the patient at the same location, with either the antigen or the CD1d ligand being administered first. The antigen and CD1d ligand may also be administered to the patient separately at different locations, e.g. in different limbs. The initial dose of CD1d ligand and antigen administered to the patient more than 1 year previously may also be administered as a single composition of the CD1d ligand and the antigen, or the CD1d ligand and antigen may have been administered sequentially or separately.

The amount of CD1d ligand administered to the patient to induce an immune response may vary depending on the age and weight of a patient to whom the composition is administered but will typically contain between 1-100 μg/kg patient bodyweight. Surprisingly, it has been found that low doses of the CD1d ligand are sufficient to enhance the immune response to a co-administered antigen and promote long-term immunological memory to that antigen. The amount of CD1d ligand included in the compositions of the invention may therefore be less than 50λg/kg patient bodyweight, less than 20 μg/kg, less than 10 μg/kg, less than 5 μg/kg, less than 4 μg/kg, or less than 3 μg/kg.

According to a further aspect of the invention, there is provided a method of inducing an immune response against an antigen in a patient comprising administering to said patient:
a) said antigen; and
b) a CD1d ligand,
   wherein the amount of CD1d ligand included in the composition is less than 10 μg/kg patient bodyweight, preferably less than 5 μg/kg, less than 4 μg/kg, or less than 3 μg/kg.

The invention also provides the use of an antigen and a CD1d ligand in the manufacture of a medicament to induce an immune response in a patient, wherein the amount of CD1d ligand is less than 10 μg/kg patient bodyweight, preferably less than 5 μg/kg, less than 4 μg/kg, or less than 3 μg/kg.

The antigen and CD1d ligand administered to the patient according to this aspect of the invention may be: administered as a mixture; administered sequentially to the patient at the same location (with either the antigen or the CD1d ligand being administered first); or administered to the patient separately at different locations, e.g. in different limbs.

### CD1d Ligands

The CD1d ligand included in the compositions of the invention may be any molecule that binds to a CD1d molecule. CD1d molecules are located on invariant NKT (iNKT) cells, B cells, dendritic cells, mononuclear cells, and conventional T cells and the CD1d ligands of the invention may bind to CD1d molecules located on any of these cells. Binding of CD1d ligands of the invention to CD1d molecules may activate iNKT cells, B cells, dendritic cells, mononuclear cells, and/or conventional T cells. Preferably, binding of CD1d ligands to CD1d molecules activates iNKT cells. The ability of a molecule to bind to a CD1d molecule may be determined by standard methods known in the art. The ability of a CD1d ligand to activate cells, in particular invariant NKT cells, may be determined by measuring the levels of cytokines released from cells in the presence of a CD1d ligand compared to the levels of cytokines released in the absence of the CD1d ligand. Preferably, the CD1d ligands included in the compositions of the invention increase the level of cytokine secretion by invariant NKT cells compared to the level of cytokine secretion by invariant NKT cells in the absence of the CD1d ligand. The CD1d ligands of the invention may promote the release of Th1 cytokines or Th2 cytokines. Preferably, the CD1d ligands of the invention increase the levels of IFN-γ, IL-4 and IL-13 secreted by invariant NKT cells compared to the levels of IFN-γ, IL-4 and IL-13 secreted by invariant NKT cells in the absence of the CD1d ligand.

Candidate molecules that may be tested for the ability to act as CD1d ligands that activate invariant NKT cells include peptides and saccharides. Preferably, the CD1d ligands of the invention are glycolipids. A review of glycolipid antigens known to act as CD1d ligands that may be included in the compositions of the invention is provided in reference 9.

Examples of suitable CD1d ligands for use in the compositions of the invention include α-glycosylceramides. α-glycosylceramides used in the compositions of the invention are preferably compounds of formula (I):

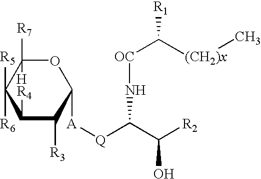

wherein
A represents O, CH₂, —CH₂CH═CH, —CH═CHCH₂,
Q represents (CH₂)ₙ wherein n represents an integer of 0 or 1,
R¹ represents H or OH,
X represents an integer between 1 and 30,
R² represents a substituent selected from the group consisting of the following (a) to (e) (wherein Y represents an integer between 5 and 17);
   (a) —CH₂(CH₂)_{Y}CH₃
   (b) —CH(OH)(CH₂)_{Y}CH₃
   (c) —CH(OH)(CH₂)_{Y}CH(CH₃)₂
   (d) —CH═CH(CH₂)_{Y}CH₃
   (e) —CH(OH)(CH₂)_{Y}CH(CH₃)CH₂CH₃,
R³ represents H, OH, NH₂, NHCOCH₃ or a monosaccharide,
R⁴ represents OH or a monosaccharide,
R⁵ represents H, OH or a monosaccharide,
R⁶ represents H, OH or a monosaccharide, and
R⁷ represents H, CH₃, CH₂OH or —CH₂-monosaccharide.

X is preferably between 7 and 27, more preferably between 9 and 24, and more preferably between 13 and 20. Y is preferably between 7 and 15, and more preferably between 9 and 13.

The term “monosaccharide” means a sugar molecule having a chain of 3-10 carbon atoms in the form of an aldehyde (aldose) or ketone (ketose). Suitable monosaccharides for use in the invention include both naturally occurring and synthetic monosaccharides. Sample monosaccharides include trioses, such as glycerose and dihydroxyacetone; textroses, such as erythanose and erythrulose; pentoses, such as xylose, arabinose, ribose, xylulose ribulose; methyl pentoses (6-deoxyhexoses), such as rhamnose and fructose; hexoses, such as glucose, mannose, galactose, fructose and sorbose; heptoses, such as glucoheptose, galamannoheptose, sedoheptulose and mannoheptulose. Preferred monosaccharides are hexoses.

The monosaccharide groups may be attached to the structure at R³, R⁴, R⁵, R⁶ or R⁷ position to form a glycosyl bond. Typically, the monosaccharide is attached to the R³, R⁴, R⁵, R⁶ or R⁷ position through the oxygen attached to the C—1 carbon of the monosaccharide, forming a glycosidic linkage.

Where R³ is a monosaccharide, it is preferably selected from α-D-galactopyranose, β-D-galactopyranose, α-D-glucopyranose or β-D-glucopyranose.

Where R⁴ is a monosaccharide, it is preferably selected from β-D-galactofuranose or N-acetyl α-D-galactopyranose.

Where R⁵ is a monosaccharide, it is preferably selected from α-D-galactopyranose, β-D-galactopyranose, α-D-glucopyranose or β-D-glucopyranose.

Where R⁶ is a monosaccharide, it is preferably selected from α-D-galactopyranose, β-D-galactopyranose, α-D-glucopyranose or β-D-glucopyranose.

Where R⁷ is a monosaccharide, it is preferably selected from methyl α-D-galactopyranoside, methyl β-D-galactopyranoside, methyl α-D-glucopyranoside or methyl β-D-glucopyranoside.

Preferably, R⁵ and R⁶ are different. Preferably, one of R⁵ and R⁶ is H.

Further examples of α-glycosylceramides suitable for inclusion in the compositions of the invention are provided in reference 2.

Preferably, the α-glycosylceramide is α-galactosylceramide (α-GalCer), having the formula given below, or an analog thereof:

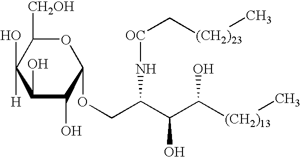

α-GalCer and analogs thereof included in the compositions of the invention may be isolated directly from marine sponges or may be chemically synthesised products.

Examples of α-GalCer analogs suitable for use in the compositions of the invention, and methods of synthesising these products, are provided in references 10 and 11. A preferred α-GalCer analog is KRN7000, which has the formula (2S,3S,4R)-1-O-(α-D-galactopyranosyl)-2-(N-hexacosanoylamino)-1,3,4-octadecanetriol. The synthesis of KRN7000 is described in reference 12.

Further preferred α-GalCer analogs are C-linked analogs of α-GalCer such as those described in references 13, 14 and 15. A preferred C-linked analog of α-GalCer is CRONY-101, the synthesis of which is described in reference 13.

Truncated analogs of α-GalCer in which the fatty acyl chain and/or the sphingosine chain are truncated compared to α-GalCer may also be used in the invention. Examples of truncated analogs of α-GalCer are provided in reference 16. A preferred truncated analog of α-GalCer is ‘OCH’ in which the fatty acyl chain has a truncation of two hydrocarbons and the sphingosine chain has a truncation of nine hydrocarbons compared to the preferred α-GalCer (i.e. R¹═H, X=21, R²═CH(OH)(CH₂)₄CH₃, R³═OH, R═OH, R⁵═OH, R⁶═H and R⁷═CH₂OH).

Further preferred truncated analogs of α-GalCer include analogs in which the fatty acyl chain has a truncation of two hydrocarbons and the sphingosine chain has a truncation of seven or three hydrocarbons compared to α-GalCer (i.e. R¹═H, X=21, R³═OH, R⁴═OH, R⁵═OH, R⁶═H, R⁷═CH₂OH and R² is either CH(OH)(CH₂)₆CH₃ or CH(OH)(CH₂)₁₀CH₃).

α-GalCer, KRN7000 and OCH are all phytosphingosine-containing α-glycosylceramides. However, the invention also includes the use of sphinganine-containing analogues of KRN700, OCH and other β-glycosylceramides described above. The synthesis of sphinganine-containing analogues of KRN7000 and OCH is described in reference 17.

CD1d ligands used in the compositions of the invention may also include sulfatide analogs, such as those described in reference 18. A preferred analog of α-GalCer 3″-O-sulfo-galactosylceramide.

Although α-GalCer was originally isolated from marine sponges, CD1d ligands of similar structure to α-GalCer have recently been isolated from Gram negative bacteria. Further CD1d ligands that may be included in the compositions of the invention are thus glycolipids of bacterial origin and in particular bacterial glycosylceramides isolated from the outer membrane of Sphingomonas and Ehilichia. Examples of such glycosylceramides include α-glucuronosylceramide and α-galacturonsylceramide from Sphingomonas, the production of which are described in reference 19 The production of further CD1d ligands from Sphingomonas and from Borrelia are described in reference 18.

The invention also includes the use of CD1d ligands that do not belong to the glycosphingolipid family. In particular, the invention includes the use of CD1d ligands that are glycoglycerol lipids. Glycoglycerol lipids that may be used in the invention include diacylglycerols, in particular monogalactosyl diacylglycerols. Suitable monogalactosyl diacyglycerols for use in the invention are described in reference 20.

### Antigenic Components of the Composition

The antigen included in the composition for inducing long-term immunological memory described above may be any antigen known for use in inducing an immune response. The antigen may comprise a protein antigen or a saccharide antigen.

### Saccharide Antigens

Where the antigen is a saccharide antigen, it is preferably conjugated to a carrier protein. Preferably, the saccharide antigen is a bacterial saccharide and in particular a bacterial capsular saccharide.

Examples of bacterial capsular saccharides which may be included in the compositions of the invention include capsular saccharides from Neisseria meningitidis (serogroups A, B, C, W135 or Y), Streptococcus pneumoniae (serotypes 4, 6B, 9V, 14, 18C, 19F or 23F), Streptococcus agalactiae (types Ia, Ib, II, III, IV, V, VI, VII, or VIII), Haemophilus influenzae (typeable strains: a, b, c, d, e or f), Pseudomonas aeruginosa, Staphylococcus aureus, etc. Other saccharides which may be included in the compositions of the invention include glucans (e.g. fungal glucans, such as those in Candida albicans), and fungal capsular saccharides e.g. from the capsule of Cryptococcus neoformans.

The N. meningitidis serogroup A (MenA) capsule is a homopolymer of (α1→6)-linked N-acetyl-D-mannosamine-1-phosphate, with partial O-acetylation in the C3 and C4 positions. The N. meningitidis serogroup B (MenB) capsule is a homopolymer of (α2→8)-linked sialic acid. The N. meningitidis serogroup C (MenC) capsular saccharide is a homopolymer of (α2→9) linked sialic acid, with variable O-acetylation at positions 7 and/or 8. The N. meningitidis serogroup W135 saccharide is a polymer consisting of sialic acid-galactose disaccharide units [→4)-D-Neup5Ac(7/9OAc)-α-(2→6)-D-Gal-α-(1→]. It has variable O-acetylation at the 7 and 9 positions of the sialic acid [21]. The N. meningitidis serogroup Y saccharide is similar to the serogroup W135 saccharide, except that the disaccharide repeating unit includes glucose instead of galactose [→4)-D-Neup5Ac(7/9OAc)-α-(2→6)-D-Glc-α-(1→]. It also has variable O-acetylation at positions 7 and 9 of the sialic acid.

The H. influenzae type b capsular (Hib) saccharide is a polymer of ribose, ribitol and phosphate [‘PRP’, (poly-3-β-D-ribose-(1.1)-D-ribitol-5-phosphate)].

The compositions of the invention may contain mixtures of saccharide antigen conjugates. Preferably, compositions of the invention comprise saccharide antigens from more than one serogroup of N. meningitidis, e.g. compositions may comprise saccharides conjugates from serogroups A+C, A+W135, A+Y, C+W135, C+Y, W135+Y, A+C+W135, A+C+Y, C+W135+Y, A+C+W135+Y, etc. Preferred compositions comprise saccharide conjugates from serogroups C and Y. Other preferred compositions comprise saccharide conjugates from serogroups C, W135 and Y.

Where a mixture comprises meningococcal saccharides from serogroup A and at least one other serogroup saccharide, the ratio (w/w) of MenA saccharide to any other serogroup saccharide may be greater than 1 (e.g. 2:1, 3:1, 4:1, 5:1, 10:1 or higher). Preferred ratios (w/w) for saccharides from serogroups A:C:W135:Y are: 1:1:1:1; 1:1:1:2; 2:1:1:1; 4:2:1:1; 8:4:2:1; 4:2:1:2; 8:4:1:2; 4:2:2:1; 2:2:1:1; 4:4:2:1; 2:2:1:2; 4:4:1:2; and 2:2:2:1.

Further preferred compositions of the invention comprise a Hib saccharide conjugate and a saccharide conjugate from at least one serogroup of N. meninigitidis, preferably from more than one serogroup of N. meningitidis. For example, a composition of the invention may comprise a Hib conjugate and cojugates from N. meningitidis serogroups A, C, W 135 and Y.

The invention further includes compositions comprising Streptococcus pneumoniae saccharide conjugates. Preferably, the compositions comprise saccharide conjugates from more than one serotype of Streptococcus pneumoniae. Preferred compositions comprise saccharide conjugates from Streptococcus pneumoniae serotypes 4, 6B, 9V, 14, 18C, 19F and 23F (7-valent). Compositions may further comprise saccharide conjugates from Streptococcus pneumoniae serotypes 4, 6B, 9V, 14, 18C, 19F 23F, 1 and 5 (9-valent) or may comprise saccharide conjugates from Streptococcus pneumoniae serotypes 4, 6B, 9V, 14, 18C, 19F, 23F, 1, 5, 3 and 7F (11-valent).

Further preferred compositions of the invention comprise pneumococcal saccharide conjugates and saccharide conjugates from Hib and/or N. meningitidis. Preferably, compositions of the invention may comprise saccharide conjugates from S. pneumoniae serotypes 4, 6B, 9V, 14, 18C, 19F and 23F and a Hib saccharide conjugate. Preferably, compositions of the invention may comprise saccharide conjugates from S. pneumoniae serotypes 4, 6B, 9V, 14, 18C, 19F and 23F and saccharide conjugates from N. meningitidis serogroups A, C, W135 and Y. Compositions according to the invention may also comprise saccharide conjugates from S. pneumoniae serotypes 4, 6B, 9V, 14, 18C, 19F and 23F, a Hib saccharide conjugate and saccharide conjugates from N. meningitidis serogroups A, C, W135 and Y.

It is preferred that the protective efficacy of individual saccharide antigen conjugates is not removed by combining them, although actual immunogenicity (e.g. ELISA titres) may be reduced.

### Preparation of Capsular Saccharide Antigens

Methods for the preparation of capsular saccharide antigens are well known. For example, ref. 22 describes the preparation of saccharide antigens from N. meningitidis. The preparation of saccharide antigens from H. influenzae is described in chapter 14 of ref. 86). The preparation of saccharide antigens and conjugates from S. pneumoniae is described in the art. For example, Prevenar™ is a 7-valent pneumococcal conjugate vaccine. Processes for the preparation of saccharide antigens from S. agalactiae is described in detail in refs. 23 and 24.

The saccharide antigens may be chemically modified. For instance, they may be modified to replace one or more hydroxyl groups with blocking groups. This is particularly useful for meningococcal serogroup A where the acetyl groups may be replaced with blocking groups to prevent hydrolysis [25]. Such modified saccharides are still serogroup A saccharides within the meaning of the present invention.

Capsular saccharides may be used in the form of oligosaccharides. These are conveniently formed by fragmentation of purified capsular polysaccharide (e.g. by hydrolysis), which will usually be followed by purification of the fragments of the desired size.

Fragmentation of polysaccharides is preferably performed to give a final average degree of polymerisation (DP) in the oligosaccharide of less than 30. DP can conveniently be measured by ion exchange chromatography or by colorimetric assays [26].

If hydrolysis is performed, the hydrolysate will generally be sized in order to remove short-length oligosaccharides [27]. This can be achieved in various ways, such as ultrafiltration followed by ion-exchange chromatography. Oligosaccharides with a degree of polymerisation of less than or equal to about 6 are preferably removed for serogroup A, and those less than around 4 are preferably removed for serogroups W135 and Y.

### Carriers

Preferably, the carrier is a protein. Preferred carrier proteins to which the saccharide antigens are conjugated in the compositions of the invention are bacterial toxins, such as diphtheria toxoid or tetanus toxoid. Suitable carrier proteins include the CRM197 mutant of diphtheria toxin [28-30], diphtheria toxoid, the N. meningitidis outer membrane protein [31], synthetic peptides [32,33], heat shock proteins [34,35], pertussis proteins [36,37], cytokines [38], lymphokines [38], hormones [38], growth factors [38], artificial proteins comprising multiple human CD4+ T cell epitopes from various pathogen-derived antigens [39] such as the N19 protein [40], protein D from H. influenzae [41,42], pneumococcal surface protein PspA [43], pneumolysin [44], iron-uptake proteins [45], toxin A or B from C. difficile [46], human serum albumin (preferably recombinant), etc.

Attachment of the saccharide antigen to the carrier is preferably via a —NH₂ group e.g. in the side chain of a lysine residue in a carrier protein, or of an arginine residue. Where a saccharide has a free aldehyde group then this can react with an amine in the carrier to form a conjugate by reductive amination. Attachment may also be via a —SH group e.g. in the side chain of a cysteine residue.

Where the composition contain more than one saccharide antigen, it is possible to use more than one carrier e.g. to reduce the risk of carrier suppression. Thus different carriers can be used for different saccharide antigens. e.g. Neisseria meningitidis serogroup A saccharides might be conjugated to CRM197 while type C saccharides might be conjugated to tetanus toxoid. It is also possible to use more than one carrier for a particular saccharide antigen. The saccharides might be in two groups, with some conjugated to CRM197 and others conjugated to tetanus toxoid. In general, however, it is preferred to use the same carrier for all saccharides.

A single carrier protein might carry more than one saccharide antigen [47,48]. For example, a single carrier protein might have conjugated to it saccharides from different pathogens or from different serogroups of the same pathogen. To achieve this goal, different saccharides can be mixed prior to the conjugation reaction. In general, however, it is preferred to have separate conjugates for each serogroup, with the different saccharides being mixed after conjugation. The separate conjugates may be based on the same carrier.

Conjugates with a saccharide:protein ratio (w/w) of between 1:5 (i.e. excess protein) and 5:1 (i.e. excess saccharide) are preferred. Ratios between 1:2 and 5:1 are preferred, as are ratios between 1:1.25 and 1:2.5.

Conjugates may be used in conjunction with free carrier [49]. When a given carrier protein is present in both free and conjugated form in a composition of the invention, the unconjugated form is preferably no more than 5% of the total amount of the carrier protein in the composition as a whole, and more preferably present at less than 2% by weight.

Any suitable conjugation reaction can be used, with any suitable linker where necessary.

The saccharide will typically be activated or functionalised prior to conjugation. Activation may involve, for example, cyanylating reagents such as CDAP (e.g. 1-cyano-4-dimethylamino pyridinium tetrafluoroborate [50, 51, etc.]). Other suitable techniques use carbodiimides, hydrazides, active esters, norborane, p-nitrobenzoic acid, N-hydroxysuccinimide, S—NHS, EDC, TSTU (see also the introduction to reference 52).

Linkages via a linker group may be made using any known procedure, for example, the procedures described in references 53 and 54. One type of linkage involves reductive amination of the polysaccharide, coupling the resulting amino group with one end of an adipic acid linker group, and then coupling a protein to the other end of the adipic acid linker group [55, 56]. Other linkers include B-propionamido [57], nitrophenyl-ethylamine [58], haloacyl halides [59], glycosidic linkages [60], 6-aminocaproic acid [61], ADH [62], C4 to C12 moieties [63] etc. As an alternative to using a linker, direct linkage can be used. Direct linkages to the protein may comprise oxidation of the polysaccharide followed by reductive amination with the protein, as described in, for example, references 64 and 65.

A process involving the introduction of amino groups into the saccharide (e.g. by replacing terminal ═O groups with —NH2) followed by derivatisation with an adipic diester (e.g. adipic acid N-hydroxysuccinimido diester) and reaction with carrier protein is preferred.

After conjugation, free and conjugated saccharides can be separated. There are many suitable methods, including hydrophobic chromatography, tangential ultrafiltration, diafiltration etc. [see also refs. 66 & 67, etc.].

Where the composition of the invention includes a depolymerised saccharide, it is preferred that depolymerisation precedes conjugation.

The preparation of suitable saccharide conjugate antigens suitable for inclusion in the compositions of the invention is described in reference 68.

### Protein Antigens

Where the antigen included in the compositions of the invention is a protein antigen, it may be selected from:
a protein antigen from N. meningitidis serogroup B, such as those in refs. 69 to 75. Using the standard nomenclature of reference 73, NMB2132, NMB1870 and NMB0992 are three preferred proteins that may be used as the basis of a suitable antigen.
a protein antigen from S. pneumoniae (e.g. from PhtA, PhtD, PhtB, PhtE, SpsA, LytB, LytC, LytA, Sp125, Sp101, Sp128, Sp130 and Sp133, as disclosed in reference 76.)
an antigen from hepatitis A virus, such as inactivated virus [e.g. 77, 78; chapter 15 of ref. 86].
an antigen from hepatitis B virus, such as the surface and/or core antigens [e.g. 78, 79; chapter 16 of ref. 86].
an antigen from hepatitis C virus [e.g. 80]. Hepatitis C virus antigens that may be used can include one or more of the following: HCV E1 and or E2 proteins, E1/E2 heterodimer complexes, core proteins and non-structural proteins, or fragments of these antigens, wherein the non-structural proteins can optionally be modified to remove enzymatic activity but retain immunogenicity (e.g. 81, 82 and 83).
an antigen from Bordetella pertussis, such as pertussis holotoxin (PT) and filamentous haemagglutinin (FHA) from B. pertussis, optionally also in combination with pertactin and/or agglutinogens 2 and 3 [e.g. refs. 84 & 85; chapter 21 of ref. 86].
a diphtheria antigen, such as a diphtheria toxoid [e.g. chapter 13 of ref. 86].
a tetanus antigen, such as a tetanus toxoid [e.g. chapter 27 of ref. 86].
an antigen from N. gonorrhoeae [e.g. 69, 70, 71].
an antigen from Chlamydia pneumoniae [e.g. 87, 88, 89, 90, 91, 92, 93].
an antigen from Chlamydia trachomatis [e.g. 94].
an antigen from Porphyromonas gingivalis [e.g. 95].
polio antigen(s) [e.g. 96, 97; chapter 24 of ref 86] such as IPV.
rabies antigen(s) [e.g. 98] such as lyophilised inactivated virus [e.g. 99, RabAvert™].
measles, mumps and/or rubella antigens [e.g. chapters 19, 20 and 26 of ref. 86].
antigens from Helicobacter pylori such as CagA [100 to 103], VacA [104, 105], NAP [106, 107, 108], HopX [e.g. 109], HopY [e.g. 109] and/or urease.
influenza antigen(s) [e.g. chapters 17 & 18 of ref. 86], such as the haemagglutinin and/or neuraminidase surface proteins.
an antigen from Moraxella catarrhalis [e.g. 110].
a protein antigen from Streptococcus agalactiae (group B streptococcus) [e.g. 111, 112].
an antigen from Streptococcus pyogenes (group A streptococcus) [e.g. 112, 113, 114].
an antigen from Staphylococcus aureus [e.g. 115].
antigen(s) from a paramyxovirus such as respiratory syncytial virus (RSV [116, 117]) and/or parainfluenza virus (PIV3 [118]).
an antigen from Bacillus anthracis [e.g. 119, 120, 121].
an antigen from a virus in the flaviviridae family (genus flavivirus), such as from yellow fever virus, Japanese encephalitis virus, four serotypes of Dengue viruses, tick-borne encephalitis virus, West Nile virus.
a pestivirus antigen, such as from classical porcine fever virus, bovine viral diarrhoea virus, and/or border disease virus.
a parvovirus antigen e.g. from parvovirus B19.
A herpes simplex virus (HSV) antigen. A preferred HSV antigen for use with the invention is membrane glycoprotein gD. It is preferred to use gD from a HSV-2 strain (‘gD2’ antigen). The composition can use a form of gD in which the C-terminal membrane anchor region has been deleted [122] e.g. a truncated gD comprising amino acids 1-306 of the natural protein with the addition of aparagine and glutamine at the C-terminus. This form of the protein includes the signal peptide which is cleaved to yield a mature 283 amino acid protein. Deletion of the anchor allows the protein to be prepared in soluble form.
a human papillomavirus (HPV) antigen. Preferred HPV antigens for use with the invention are L1 capsid proteins, which can assemble to form structures known as virus-like particles (VLPs). The VLPs can be produced by recombinant expression of L1 in yeast cells (e.g. in S. cerevisiae) or in insect cells (e.g. in Spodoptera cells, such as S. frugiperda, or in Drosophila cells). For yeast cells, plasmid vectors can carry the L1 gene(s); for insect cells, baculovirus vectors can carry the L1 gene(s). More preferably, the composition includes L1 VLPs from both HPV-16 and HPV-18 strains. This bivalent combination has been shown to be highly effective [123]. In addition to HPV-16 and HPV-18 strains, it is also possible to include L1 VLPs from HPV-6 and HPV-11 strains. The use of oncogenic HPV strains is also possible. A vaccine may include between 20-60 μg/ml (e.g. about 40 μg/ml) of L1 per HPV strain.

The composition may comprise one or more of these antigens, which may be detoxified where necessary (e.g. detoxification of pertussis toxin by chemical and/or genetic means).

Where a diphtheria antigen is included in the mixture it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens.

Antigens in the mixture will typically be present at a concentration of at least 1 μg/ml each. In general, the concentration of any given antigen will be sufficient to elicit an immune response against that antigen.

As an alternative to using proteins antigens in the mixture, nucleic acid encoding the antigen may be used. Protein components of the mixture may thus be replaced by nucleic acid (preferably DNA e.g. in the form of a plasmid) that encodes the protein. Similarly, compositions of the invention may comprise proteins which mimic antigens e.g. mimotopes [124] or anti-idiotype antibodies.

Alternatively or in addition to the antigens listed above, the composition may comprise an outer-membrane vesicle (OMV) preparation from N. meningitidis serogroup B, such as those disclosed in refs. 125, 126, 127, 128, etc.

### Additional Compositions

A further object of the invention is to provide vaccine compositions that provide protection against group B streptococcus, N. meningitidis serogroup B and/or influenza virus. It has been found that CD1d ligands are surprisingly effective adjuvants for antigens from these pathogens.

The compositions described below include at least one antigen from group B Streptococcus, N. meningitidis serogroup B or influenza virus. These compositions may comprise additional antigens. For example, these compositions may also include one or more saccharide antigens conjugated to one or more carriers such as those described above for inclusion in compositions for use in inducing long-term immunological memory. Alternatively, or in addition, these compositions may comprise one or more of the protein antigens described above.

Group B streptococcus

The invention therefore provides a composition comprising: a) a CD1d ligand; and b) an antigen from group B streptococcus.

Examples of antigens from group B streptococcus (Streptococcus agalactiae) for inclusion in the composition are found in references 111 & 112. Thus the composition may include a protein comprising one or more of: (i) the S. agalactiae amino acid sequences in ref. 112 (even-numbered SEQ ID NOs: 2 to 10960 of ref. 112); (ii) an amino acid sequence having at least 80% sequence identity to a S. agalactiae amino acid sequence of (i); an amino acid sequence comprising an epitope of from a S. agalactiae amino acid sequence of (i). Preferably, the composition comprises one or more of the GBS1 to GBS689 proteins as described in reference 112 (see Table IV therein). More preferably, the composition comprises a GBS80 protein antigen.

### Meningococcus

The invention also provides a composition comprising: a) a CD1d ligand; and b) an antigen from Neisseria meningitidis.

The antigen from N. meningitidis included in the composition may be a protein antigen or an outer membrane vesicle (OMV) preparation. Examples of OMV preparations that may be included in the composition include OMV preparations from N. meningitidis serogroup A, B, C, W135, or Y. Examples of protein antigens from N. meningitidis that may be included in the composition are also provided above. Preferably, the protein antigen is derived from N. meningitidis serogroup B and that, when administered to a patient, elicits an immune response that cross-reacts with N. meningitidis serogroup B cells. Preferred protein antigens that elicit an immune response that cross-reacts with N. meningitidis serogroup B cells include the ‘ΔG287nz-953’, ‘936-741’ and ‘961c’ protein antigens [129]. Preferably, the composition comprises more than one antigen from N. meningitidis. Preferably, the composition comprises all three ‘ΔG287nz-953’, ‘936-741’ and ‘961c’ protein antigens. Other useful protein antigens are based on NMB2132, NMB1870 and NMB0992.

### Influenza Virus

The invention also provides a composition comprising: a) a CD1d ligand; and b) an influenza virus antigen.

The influenza virus antigen will typically be prepared from influenza virions but, as an alternative, antigens such as haemagglutinin can be expressed in a recombinant host (e.g. in an insect cell line using a baculovirus vector) and used in purified form [130,131]. In general, however, antigens will be from virions.

The antigen may take the form of a live virus or, more preferably, an inactivated virus. Where an inactivated virus is used, the vaccine may comprise whole virion, split virion, or purified surface antigens (including hemagglutinin and, usually, also including neuraminidase). Influenza antigens can also be presented in the form of virosomes [132].

The influenza virus may be attenuated. The influenza virus may be temperature-sensitive. The influenza virus may be cold-adapted. These three possibilities apply in particular for live viruses.

Influenza virus strains for use in vaccines change from season to season. In the current inter-pandemic period, vaccines typically include two influenza A strains (H1N1 and H3N2) and one influenza B strain, and trivalent vaccines are typical. The invention may also use viruses from pandemic strains (i.e. strains to which the vaccine recipient and the general human population are immunologically naïve), such as H2, H5, H7 or H9 subtype strains (in particular of influenza A virus), and influenza vaccines for pandemic strains may be monovalent or may be based on a normal trivalent vaccine supplemented by a pandemic strain. Depending on the season and on the nature of the antigen included in the vaccine, however, the invention may protect against one or more of HA subtypes H1, H2, H3, H4, H5, H6, H7, H8, H9, H10, H11, H12, H13, H14, H15 or H16.

Other strains that can usefully be included in the compositions are strains which are resistant to antiviral therapy (e.g. resistant to oseltamivir [133] and/or zanamivir), including resistant pandemic strains [134].

The adjuvanted compositions of the invention are particularly useful for immunizing against pandemic strains. The characteristics of an influenza strain that give it the potential to cause a pandemic outbreak are: (a) it contains a new hemagglutinin compared to the hemagglutinins in currently-circulating human strains, i.e. one that has not been evident in the human population for over a decade (e.g. H2), or has not previously been seen at all in the human population (e.g. H5, H6 or H9, that have generally been found only in bird populations), such that the human population will be immunologically naïve to the strain's hemagglutinin; (b) it is capable of being transmitted horizontally in the human population; and (c) it is pathogenic to humans. A virus with H5 haemagglutinin type is preferred for immunising against pandemic influenza, such as a H5N1 strain. Other possible strains include H5N3, H9N2, H2N2, H7N1 and H7N7, and any other emerging potentially pandemic strains.

Compositions of the invention may include antigen(s) from one or more (e.g. 1, 2, 3, 4 or more) influenza virus strains, including influenza A virus and/or influenza B virus. Where a vaccine includes more than one strain of influenza, the different strains are typically grown separately and are mixed after the viruses have been harvested and antigens have been prepared. Thus a process of the invention may include the step of mixing antigens from more than one influenza strain.

The influenza virus may be a reassortant strain, and may have been obtained by reverse genetics techniques. Reverse genetics techniques [e.g. 135-139] allow influenza viruses with desired genome segments to be prepared in vitro using plasmids. Typically, it involves expressing (a) DNA molecules that encode desired viral RNA molecules e.g. from polI promoters, and (b) DNA molecules that encode viral proteins e.g. from polII promoters, such that expression of both types of DNA in a cell leads to assembly of a complete intact infectious virion. The DNA preferably provides all of the viral RNA and proteins, but it is also possible to use a helper virus to provide some of the RNA and proteins. Plasmid-based methods using separate plasmids for producing each viral RNA are preferred [140-142], and these methods will also involve the use of plasmids to express all or some (e.g. just the PB1, PB2, PA and NP proteins) of the viral proteins, with up to 12 plasmids being used in some methods. To reduce the number of plasmids needed, a recent approach [143] combines a plurality of RNA polymerase I transcription cassettes (for viral RNA synthesis) on the same plasmid (e.g. sequences encoding 1, 2, 3, 4, 5, 6, 7 or all 8 influenza A vRNA segments), and a plurality of protein-coding regions with RNA polymerase II promoters on another plasmid (e.g. sequences encoding 1, 2, 3, 4, 5, 6, 7 or all 8 influenza A mRNA transcripts). Preferred aspects of the reference 143 method involve: (a) PB1, PB2 and PA mRNA-encoding regions on a single plasmid; and (b) all 8 vRNA-encoding segments on a single plasmid. It is possible to use dual polI and polII promoters to simultaneously code for the viral RNAs and for expressible mRNAs from a single template [144,145].

Thus the virus may include one or more RNA segments from a A/PR/8/34 virus (typically 6 segments from A/PR/8/34, with the HA and N segments being from a vaccine strain, i.e. a 6:2 reassortant), particularly when viruses are grown in eggs. It may also include one or more RNA segments from a A/WSN/33 virus, or from any other virus strain useful for generating reassortant viruses for vaccine preparation. Typically, the invention protects against a strain that is capable of human-to-human transmission, and so the strain's genome will usually include at least one RNA segment that originated in a mammalian (e.g. in a human) influenza virus.

The viruses used as the source of the antigens can be grown either on eggs or on cell culture. The current standard method for influenza virus growth uses embryonated hen eggs, with virus being purified from the egg contents (allantoic fluid). More recently, however, viruses have been grown in animal cell culture and, for reasons of speed and patient allergies, this growth method is preferred.

The cell substrate will typically be a mammalian cell line, such as MDCK; CHO; 293T; BHK; Vero; MRC-5; PER.C6; WI-38; etc. Preferred mammalian cell lines for growing influenza viruses include: MDCK cells [146-149], derived from Madin Darby canine kidney; Vero cells [150-152], derived from African green monkey (Cercopithecus aethiops) kidney; or PER.C6 cells [153], derived from human embryonic retinoblasts. These cell lines are widely available e.g. from the American Type Cell Culture (ATCC) collection [154], from the Coriell Cell Repositories [155], or from the European Collection of Cell Cultures (ECACC). For example, the ATCC supplies various different Vero cells under catalog numbers CCL-81, CCL-81.2, CRL-1586 and CRL-1587, and it supplies MDCK cells under catalog number CCL-34. PER.C6 is available from the ECACC under deposit number 96022940. As a less-preferred alternative to mammalian cell lines, virus can be grown on avian cell lines [e.g. refs. 156-158], including cell lines derived from ducks (e.g. duck retina) or hens e.g. chicken embryo fibroblasts (CEF), etc.

Where virus has been grown on a mammalian cell line then the composition will advantageously be free from egg proteins (e.g. ovalbumin and ovomucoid) and from chicken DNA, thereby reducing allergenicity.

For growth on a cell line, such as on MDCK cells, virus may be grown on cells in suspension [146] or in adherent culture. One suitable MDCK cell line for suspension culture is MDCK 33016 (deposited as DSM ACC 2219). As an alternative, microcarrier culture can be used.

Where virus has been grown on a cell line then the culture for growth will preferably be free from (i.e. will have been tested for and given a negative result for contamination by) herpes simplex virus, respiratory syncytial virus, parainfluenza virus 3, SARS coronavirus, adenovirus, rhinovirus, reoviruses, polyomaviruses, birnaviruses, circoviruses, and/or parvoviruses. Absence of herpes simplex viruses is particularly preferred.

Where virus has been grown on a cell line then the composition preferably contains less than 10 ng (preferably less than 1 ng, and more preferably less than 100 pg) of residual host cell DNA per dose, although trace amounts of host cell DNA may be present. In general, the host cell DNA that it is desirable to exclude from compositions of the invention is DNA that is longer than 100 bp.

Measurement of residual host cell DNA is now a routine regulatory requirement for biologicals and is within the normal capabilities of the skilled person. The assay used to measure DNA will typically be a validated assay [159,160]. The performance characteristics of a validated assay can be described in mathematical and quantifiable terms, and its possible sources of error will have been identified. The assay will generally have been tested for characteristics such as accuracy, precision, specificity. Once an assay has been calibrated (e.g. against known standard quantities of host cell DNA) and tested then quantitative DNA measurements can be routinely performed. Three principle techniques for DNA quantification can be used: hybridization methods, such as Southern blots or slot blots [161]; immunoassay methods, such as the Threshold™ System [162]; and quantitative PCR [163]. These methods are all familiar to the skilled person, although the precise characteristics of each method may depend on the host cell in question e.g. the choice of probes for hybridization, the choice of primers and/or probes for amplification, etc. The Threshold™ system from Molecular Devices is a quantitative assay for picogram levels of total DNA, and has been used for monitoring levels of contaminating DNA in biopharmaceuticals [162]. A typical assay involves non-sequence-specific formation of a reaction complex between a biotinylated ssDNA binding protein, a urease-conjugated anti-ssDNA antibody, and DNA. All assay components are included in the complete Total DNA Assay Kit available from the manufacturer. Various commercial manufacturers offer quantitative PCR assays for detecting residual host cell DNA e.g. AppTec™ Laboratory Services, BioReliance™, Althea Technologies, etc. A comparison of a chemiluminescent hybridisation assay and the total DNA Threshold™ system for measuring host cell DNA contamination of a human viral vaccine can be found in reference 164.

Contaminating DNA can be removed during vaccine preparation using standard purification procedures e.g. chromatography, etc. Removal of residual host cell DNA can be enhanced by nuclease treatment e.g. by using a DNase. A convenient method for reducing host cell DNA contamination is disclosed in references 165 & 166, involving a two-step treatment, first using a DNase (e.g. Benzonase) and then a cationic detergent (e.g. CTAB).

Vaccines containing <10 ng (e.g. <1 ng, <100 pg) host cell DNA per 15 μg of haemagglutinin are preferred, as are vaccines containing <long (e.g. <1 ng, <100 pg) host cell DNA per 0.25 ml volume. Vaccines containing <10 ng (e.g. <1 ng, <100 pg) host cell DNA per 50 μg of haemagglutinin are more preferred, as are vaccines containing <long (e.g. <1 ng, <100 pg) host cell DNA per 0.5 ml volume.

Cell lines supporting influenza virus replication are preferably grown in serum-free culture media and/or protein free media. A medium is referred to as a serum-free medium in the context of the present invention in which there are no additives from serum of human or animal origin. Protein-free is understood to mean cultures in which multiplication of the cells occurs with exclusion of proteins, growth factors, other protein additives and non-serum proteins, but can optionally include proteins such as trypsin or other proteases that may be necessary for viral growth. The cells growing in such cultures naturally contain proteins themselves.

Cell lines supporting influenza virus replication are preferably grown below 37° C. [167] e.g. 30-36° C.

Haemagglutinin (HA) is the main immunogen in inactivated influenza vaccines, and vaccine doses are standardised by reference to HA levels, typically as measured by a single radial immunodiffution (SRID) assay. Vaccines typically contain about 15 μg of HA per strain, although lower doses are also used e.g. for children, or in pandemic situations. Fractional doses such as ½ (i.e. 7.5 μg HA per strain), ¼ and ⅛ have been used [168,169] as have higher doses (e.g. 3× or 9× doses [170,171]). Thus vaccines may include between 0.1 and 150 μg of HA per influenza strain, preferably between 0.1 and 50 μg e.g. 0.1-20 μg, 0.1-15 μg, 0.1-10 μg, 0.1-7.5 μg, 0.5-5 μg, etc. Particular doses include e.g. about 15, about 10, about 7.5, about 5, about 3.8, about 1.9, about 1.5, etc. per strain. Thus vaccines may include between 0.1 and 20 μg of HA per influenza strain, preferably between 0.1 and 15 μg e.g. 0.1-10 μg, 0.1-7.5 μg, 0.5-5 μg, etc. Particular doses include e.g. about 15, about 10, about 7.5, about 5, about 3.8, about 1.9, etc. These lower doses are most useful when an adjuvant is present in the vaccine, as with the invention.

HA used with the invention may be a natural HA as found in a virus, or may have been modified. For instance, it is known to modify HA to remove determinants (e.g. hyper-basic regions) that cause a virus to be highly pathogenic in avian species, as these determinants can otherwise prevent a virus from being grown in eggs.

An inactivated but non-whole cell vaccine (e.g. a split virus vaccine or a purified surface antigen vaccine) may include matrix protein, in order to benefit from the additional T cell epitopes that are located within this antigen. Thus a non-whole cell vaccine (particularly a split vaccine) that includes haemagglutinin and neuraminidase may additionally include M1 and/or M2 matrix protein. Where a matrix protein is present, inclusion of detectable levels of M2 matrix protein is preferred. Nucleoprotein may also be present.

### Formulation of Pharmaceutical Compositions

The antigens and CD1d ligands described above are particularly suited to inclusion in immunogenic compositions and vaccines. A process of the invention may therefore include the step of formulating an antigen and CD1d ligand as an immunogenic composition or vaccine. The invention provides a composition or vaccine obtainable in this way.

Immunogenic compositions and vaccines of the invention will, in addition to antigen(s) and CD1d ligands, typically comprise ‘pharmaceutically acceptable carriers’, which include any carrier that does not itself induce the production of antibodies harmful to the individual receiving the composition. Suitable carriers are typically large, slowly metabolised macromolecules such as proteins, polysaccharides, polylactic acids, polyglycolic acids, polymeric amino acids, amino acid copolymers, trehalose [172], lipid aggregates (such as oil droplets or liposomes), and inactive virus particles. Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. A thorough discussion of pharmaceutically acceptable excipients is available in ref. 173.

Immunogenic compositions used as vaccines comprise an immunologically effective amount of antigen, as well as any other of the above-mentioned components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

The vaccine may be administered in conjunction with other immunoregulatory agents. The CD1d ligand acts as an adjuvant within the immunogenic compositions of the invention. The vaccine may include additional adjuvants. Such adjuvants include, but are not limited to:

Adjuvants that can be used with the invention include, but are not limited to:
A mineral-containing composition, including calcium salts and aluminum salts (or mixtures thereof). Calcium salts include calcium phosphate (e.g. the “CAP” particles disclosed in ref. 174). Aluminum salts include hydroxides, phosphates, sulfates, etc., with the salts taking any suitable form (e.g. gel, crystalline, amorphous, etc.). Adsorption to these salts is preferred. The mineral containing compositions may also be formulated as a particle of metal salt [175]. Aluminum salt adjuvants are described in more detail below.
An oil-in-water emulsion, as described in more detail below.
An immunostimulatory oligonucleotide, such as one containing a CpG motif (a dinucleotide sequence containing an unmethylated cytosine linked by a phosphate bond to a guanosine), a TpG motif [176], a double-stranded RNA, an oligonucleotide containing a palindromic sequence, or an oligonucleotide containing a poly(dG) sequence. Immunostimulatory oligonucleotides can include nucleotide modifications/analogs such as phosphorothioate modifications and can be double-stranded or (except for RNA) single-stranded. References 177 to 179 disclose possible analog substitutions e.g. replacement of guanosine with 2′-deoxy-7-deazaguanosine. The adjuvant effect of CpG oligonucleotides is further discussed in refs. 180-185. A CpG sequence may be directed to TLR9, such as the motif GTCGTT or TTCGTT [186]. The CpG sequence may be specific for inducing a Th1 immune response, such as a CPG-A ODN (oligodeoxynucleotide), or it may be more specific for inducing a B cell response, such a CPG-B ODN. CpG-A and CPG-B ODNs are discussed in refs. 187-189. Preferably, the CpG is a CPG-A ODN. Preferably, the CpG oligonucleotide is constructed so that the 5′ end is accessible for receptor recognition. Optionally, two CpG oligonucleotide sequences may be attached at their 3′ ends to form “immunomers”. See, for example, references 190-192. A useful CpG adjuvant is CpG7909, also known as ProMune™ (Coley Pharmaceutical Group, Inc.). Immunostimulatory oligonucleotides will typically comprise at least 20 nucleotides. They may comprise fewer than 100 nucleotides.
3-O-deacylated monophosphoryl lipid A (‘3dMPL’, also known as ‘MPL™’) [193-196]. 3dMPL has been prepared from a heptoseless mutant of Salmonella minnesota, and is chemically similar to lipid A but lacks an acid-labile phosphoryl group and a base-labile acyl group. Preparation of 3dMPL was originally described in reference 197. 3dMPL can take the form of a mixture of related molecules, varying by their acylation (e.g. having 3, 4, 5 or 6 acyl chains, which may be of different lengths). The two glucosamine (also known as 2-deoxy-2-amino-glucose) monosaccharides are N-acylated at their 2-position carbons (i.e. at positions 2 and 2′), and there is also O-acylation at the 3′ position.
An imidazoquinoline compound, such as Imiquimod (“R-837”) [198,199], Resiquimod (“R-848”) [200], and their analogs; and salts thereof (e.g. the hydrochloride salts). Further details about immunostimulatory imidazoquinolines can be found in references 201 to 205.
A thiosemicarbazone compound, such as those disclosed in reference 206. Methods of formulating, manufacturing, and screening for active compounds are also described in reference 206. The thiosemicarbazones are particularly effective in the stimulation of human peripheral blood mononuclear cells for the production of cytokines, such as TNF-α.
A tryptanthrin compound, such as those disclosed in reference 207. Methods of formulating, manufacturing, and screening for active compounds are also described in reference 207. The thiosemicarbazones are particularly effective in the stimulation of human peripheral blood mononuclear cells for the production of cytokines, such as TNF-α.
A nucleoside analog, such as: (a) Isatorabine (ANA-245; 7-thia-8-oxoguanosine):

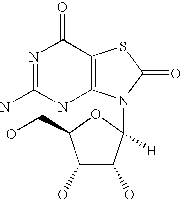

and prodrugs thereof; (b) ANA975; (c) ANA-025-1; (d) ANA380; (e) the compounds disclosed in references 208 to 210; (f) a compound having the formula:

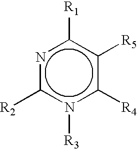

wherein:
   R₁ and R₂ are each independently H, halo, —NRₐR_{b}, —OH, C₁₋₆ alkoxy, substituted C₁₋₆ alkoxy, heterocyclyl, substituted heterocyclyl, C₆₋₁₀ aryl, substituted C₆₋₁₀ aryl, C₁₋₄ alkyl, or substituted C₁₋₆ alkyl;
   R₃ is absent, H, C₁₋₆ alkyl, substituted C₁₋₆ alkyl, C₆₋₁₀ aryl, substituted C₆₋₁₀ aryl, heterocyclyl, or substituted heterocyclyl;
   R₄ and R₅ are each independently H, halo, heterocyclyl, substituted heterocyclyl, —C(O)—R_{d}, C₁₋₆ alkyl, substituted C₁₋₆ alkyl, or bound together to form a 5 membered ring as in R₄₋₅:

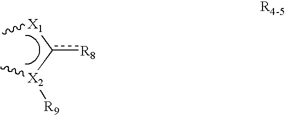

the binding being achieved at the bonds indicated by a [symbol]
X₁ and X₂ are each independently N, C, O, or S;
R₈ is H, halo, —OH, C₁₋₆ alkyl, C₂₋₆ alkenyl, C₂₋₆ alkynyl, —OH, —NRₐR_{b}, —(CH₂)ₙ—O—R_{c}, —O—(C₁₋₆ alkyl), —S(O)ₚ, or —C(O)—R_{d};
R₉ is H, C₁₋₆ alkyl, substituted C₁₋₆ alkyl, heterocyclyl, substituted heterocyclyl or R₉ₐ, wherein R₉ₐ is:

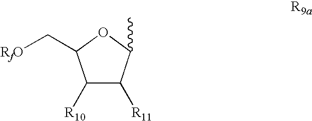

the binding being achieved at the bond indicated by a [symbol]
R₁₀ and R₁₁, are each independently H, halo, C₁₋₆ alkoxy, substituted C₁₋₆ alkoxy, —NRₐR_{b}, or —OH;
each Rₐ and R_{b} is independently H, C₁₋₆ alkyl, substituted C₁₋₆ alkyl, —C(O)R_{d}, C₆₋₁₀ aryl;
each R_{C} is independently H, phosphate, diphosphate, triphosphate, C₁₋₆ alkyl, or substituted C₁₋₆ alkyl;
each R_{d} is independently H, halo, C₁₋₆ alkyl, substituted C₁₋₆ alkyl, C₁₋₆ alkoxy, substituted C₁₋₆ alkoxy, —NH₂, —NH(C₁₋₆ alkyl), —NH(substituted C₁₋₆ alkyl), —N(C₁₋₆ alkyl)₂, —N(substituted C₁₋₆ alkyl)₂, C₆₋₁₀ aryl, or heterocyclyl;
each Rₑ is independently H, C₁₋₆ alkyl, substituted C₁₋₆ alkyl, C₆₋₁₀ aryl, substituted C₆₋₁₀ aryl, heterocyclyl, or substituted heterocyclyl;
each R_{f} is independently H, C₁₋₆ alkyl, substituted C₁₋₆ alkyl, —C(O)R_{d}, phosphate, diphosphate, or triphosphate;
each n is independently 0, 1, 2, or 3;
each p is independently 0, 1, or 2; or
or (g) a pharmaceutically acceptable salt of any of (a) to (f), a tautomer of any of (a) to (f), or a pharmaceutically acceptable salt of the tautomer.
Loxoribine (7-allyl-8-oxoguanosine) [211].
Compounds disclosed in reference 212, including: Acylpiperazine compounds, Indoledione compounds, Tetrahydraisoquinoline (THIQ) compounds, Benzocyclodione compounds, Aminoazavinyl compounds, Aminobenzimidazole quinolinone (ABIQ) compounds [213,214], Hydrapthalamide compounds, Benzophenone compounds, Isoxazole compounds, Sterol compounds, Quinazilinone compounds, Pyrrole compounds [215], Anthraquinone compounds, Quinoxaline compounds, Triazine compounds, Pyrazalopyrimidine compounds, and Benzazole compounds [216].
Compounds disclosed in reference 217, including 3,4-di(1H-indol-3-yl)-1H-pyrrole-2,5-diones, staurosporine analogs, derivatized pyridazines, chromen-4-ones, indolinones, quinazolines, and nucleoside analogs.
An aminoalkyl glucosaminide phosphate derivative, such as RC-529 [218,219].
A phosphazene, such as poly[di(carboxylatophenoxy)phosphazene] (“PCPP”) as described, for example, in references 220 and 221.
Small molecule immunopotentiators (SMIPs) such as:
N2-methyl-1-(2-methylpropyl)-1H-imidazo[4,5-c]quinoline-2,4-diamine
N2,N2-dimethyl-1-(2-methylpropyl)-1H-imidazo[4,5-c]quinoline-2,4-diamine
N2-ethyl-N2-methyl-1-(2-methylpropyl)-1H-imidazo[4,5-c]quinoline-2,4-diamine
N2-methyl-1-(2-methylpropyl)-N2-propyl-1H-imidazo[4,5-c]quinoline-2,4-diamine
1-(2-methylpropyl)-N2-propyl-1H-imidazo[4,5-c]quinoline-2,4-diamine
N2-butyl-1-(2-methylpropyl)-1H-imidazo[4,5-c]quinoline-2,4-diamine
N2-butyl-N2-methyl-1-(2-methylpropyl)-1H-imidazo[4,5-c]quinoline-2,4-diamine
N2-methyl-1-(2-methylpropyl)-N2-pentyl-1H-imidazo[4,5-c]quinoline-2,4-diamine
N2-methyl-1-(2-methylpropyl)-N2-prop-2-enyl-1H-imidazo[4,5-c]quinoline-2,4-diamine
1-(2-methylpropyl)-2-[(phenylmethyl)thio]-1H-imidazo[4,5-c]quinolin-4-amine
1-(2-methylpropyl)-2-(propylthio)-1H-imidazo[4,5-c]quinolin-4-amine
2-[[4-amino-1-(2-methylpropyl)-1H-imidazo[4,5-c]quinolin-2-yl](methyl)amino]ethanol
2-[[4-amino-1-(2-methylpropyl)-1H-imidazo[4,5-c]quinolin-2-yl](methyl)amino]ethyl acetate
4-amino-1-(2-methylpropyl)-1,3-dihydro-2H-imidazo[4,5-c]quinolin-2-one
N2-butyl-1-(2-methylpropyl)-N4,N4-bis(phenylmethyl)-1H-imidazo[4,5-c]quinoline-2,4-diamine
N2-butyl-N2-methyl-1-(2-methylpropyl)-N4,N4-bis(phenylmethyl)-1H-imidazo[4,5-c]quinoline-2,4-diamine
N2-methyl-1-(2-methylpropyl)-N4,N4-bis(phenylmethyl)-1H-imidazo[4,5-c]quinoline-2,4-diamine
N2,N2-dimethyl-1-(2-methylpropyl)-N4,N4-bis(phenylmethyl)-1H-imidazo[4,5-c]quinoline-2,4-diamine
1-{4-amino-2-[methyl(propyl)amino]-1H-imidazo[4,5-c]quinolin-1-yl}-2-methylpropan-2-ol
1-[4-amino-2-(propylamino)-1H-imidazo[4,5-c]quinolin-1-yl]-2-methylpropan-2-ol
N4,N4-dibenzyl-1-(2-methoxy-2-methylpropyl)-N2-propyl-1H-imidazo[4,5-c]quinoline-2,4-diamine.
   Saponins [chapter 22 of ref. 249], which are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis(soap root). Saponin adjuvant formulations include purified formulations, such as QS21, as well as lipid formulations, such as ISCOMs. QS21 is marketed as Stimulon™. Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 222. Saponin formulations may also comprise a sterol, such as cholesterol [223]. Combinations of saponins and cholesterols can be used to form unique particles called immunostimulating complexs (ISCOMs) [chapter 23 of ref. 249]. ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in refs. 223-225. Optionally, the ISCOMS may be devoid of additional detergent [226]. A review of the development of saponin based adjuvants can be found in refs. 227 & 228.
   Bacterial ADP-ribosylating toxins (e.g. the E. coli heat labile enterotoxin “LT”, cholera toxin “CT”, or pertussis toxin “PT”) and detoxified derivatives thereof, such as the mutant toxins known as LT-K63 and LT-R72 [229]. The use of detoxified ADP-ribosylating toxins as mucosal adjuvants is described in ref. 230 and as parenteral adjuvants in ref. 231.
   Bioadhesives and mucoadhesives, such as esterified hyaluronic acid microspheres [232] or chitosan and its derivatives [233].
   Microparticles (i.e. a particle of ˜100 nm to ˜150 μm in diameter, more preferably ˜200 nm to ˜30 μm in diameter, or ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) being preferred, optionally treated to have a negatively-charged surface (e.g. with SDS) or a positively-charged surface (e.g. with a cationic detergent, such as CTAB).
   Liposomes (Chapters 13 & 14 of ref. 249). Examples of liposome formulations suitable for use as adjuvants are described in refs. 234-236.
   Polyoxyethylene ethers and polyoxyethylene esters [237]. Such formulations further include polyoxyethylene sorbitan ester surfactants in combination with an octoxynol [238] as well as polyoxyethylene alkyl ethers or ester surfactants in combination with at least one additional non-ionic surfactant such as an octoxynol [239]. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.
   Muramyl peptides, such as N-acetylmuramyl-L-threonyl-D-isoglutamine (“thr-MDP”), N-acetyl-normuramyl-L-alanyl-D-isoglutamine (nor-MDP), N-acetylglucsaminyl-N-acetylmuramyl-L-Al-D-isoglu-L-Ala-dipalmitoxy propylamide (“DTP-DPP”, or “Theramide™), N-acetylmuramyl-L-alanyl-D-isoglutaminyl-L-alanine-2-(1′-2′dipalmitoyl-sn-glycero-3-hydroxyphosphoryloxy)-ethylamine (“MTP-PE”).
   An outer membrane protein proteosome preparation prepared from a first Gram-negative bacterium in combination with a liposaccharide (LPS) preparation derived from a second Gram-negative bacterium, wherein the outer membrane protein proteosome and LPS preparations form a stable non-covalent adjuvant complex. Such complexes include “IVX-908”, a complex comprised of Neisseria meningitidis outer membrane and LPS.
   Methyl inosine 5′-monophosphate (“MIMP”) [240].
   A polyhydroxlated pyrrolizidine compound [241], such as one having formula:

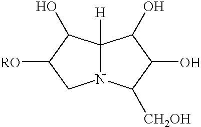

where R is selected from the group comprising hydrogen, straight or branched, unsubstituted or substituted, saturated or unsaturated acyl, alkyl (e.g. cycloalkyl), alkenyl, alkynyl and aryl groups, or a pharmaceutically acceptable salt or derivative thereof. Examples include, but are not limited to: casuarine, casuarine-6-α-D-glucopyranose, 3-epi-casuarine, 7-epi-casuarine, 3,7-diepi-casuarine, etc.
A gamma inulin [242] or derivative thereof, such as algammulin.
A compound of formula I, II or III, or a salt thereof:

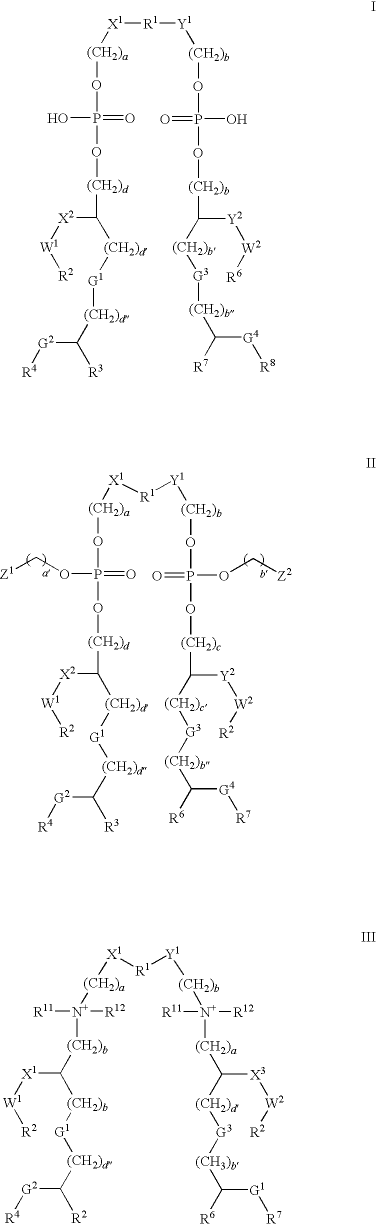

as defined in reference 243, such as ‘ER 803058’, ‘ER 803732’, ‘ER 804053’, ‘ER 804058’, ‘ER 804059’, ‘ER 804442’, ‘ER 804680’, ‘ER 804764’, ER 803022 or ‘ER 804057’ e.g.:

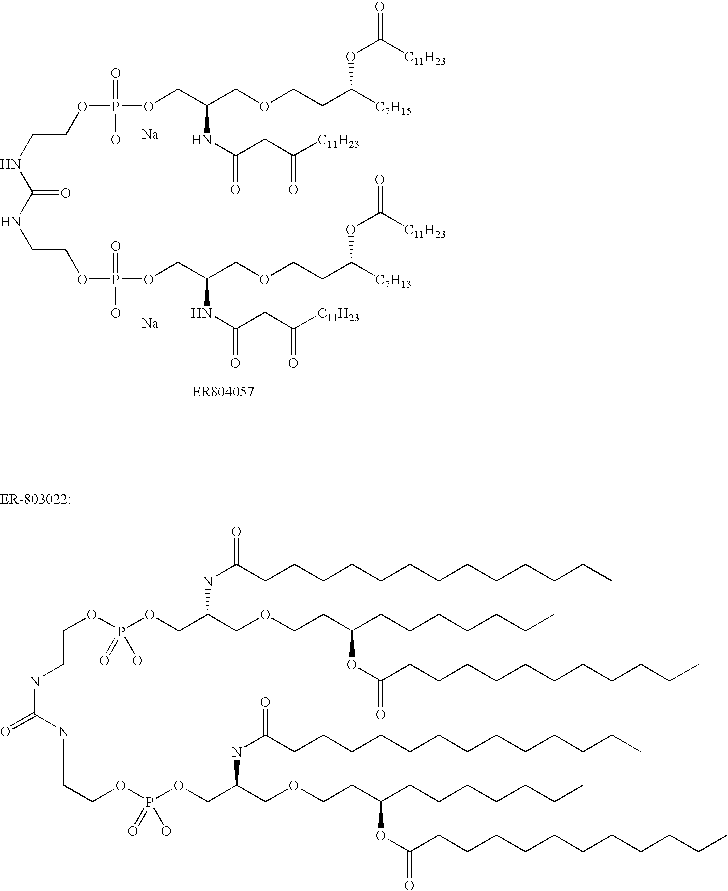

Derivatives of lipid A from Escherichia coli such as OM-174 (described in refs. 244 & 245).
A formulation of a cationic lipid and a (usually neutral) co-lipid, such as aminopropyl-dimethyl-myristoleyloxy-propanaminium bromide-diphytanoylphosphatidyl-ethanolamine (“Vaxfectin™”) or aminopropyl-dimethyl-bis-dodecyloxy-propanaminium bromide-dioleoylphosphatidyl-ethanolamine (“GAP-DLRIE:DOPE”). Formulations containing (±) —N-(3-aminopropyl)-N,N-dimethyl-2,3-bis(syn-9-tetradeceneyloxy)-1-propanaminium salts are preferred [246].
Compounds containing lipids linked to a phosphate-containing acyclic backbone, such as the TLR4 antagonist E5564 [247,248]:

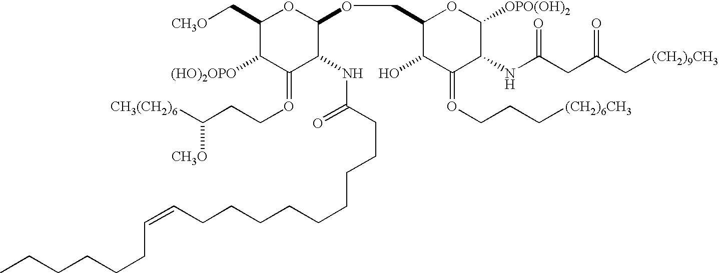

These and other adjuvant-active substances are discussed in more detail in references 249 & 250.

### Medical Methods and Uses

Once formulated, the compositions of the invention can be administered directly to the subject. The subjects to be treated can be animals; in particular, human subjects can be treated. The vaccines are particularly useful for vaccinating children and teenagers. The vaccines have been shown to be effective in MHC II−/− animal models and it is therefore considered that they will be useful for treating immunocompromised subjects. They may be delivered by systemic and/or mucosal routes.

Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect. Direct delivery of the compositions will generally be parenteral (e.g. by injection, either subcutaneously, intraperitoneally, intravenously or intramuscularly or delivered to the interstitial space of a tissue). The compositions can also be administered into a lesion. Other modes of administration include oral and pulmonary administration, suppositories, and transdermal or transcutaneous applications (e.g. see ref. 251), needles, and hyposprays. Dosage treatment may be a single dose schedule or a multiple dose schedule (e.g. including booster doses).

Vaccines of the invention are preferably sterile. They are preferably pyrogen-free. They are preferably buffered e.g. at between pH 6 and pH 8, generally around pH 7.

Vaccines of the invention may comprise detergent (e.g. a Tween, such as Tween 80) at low levels (e.g. <0.01%). Vaccines of the invention may comprise a sugar alcohol (e.g. mannitol) or trehalose e.g. at around 15 mg/ml, particularly if they are to be lyophilised.

Optimum doses of individual antigens can be assessed empirically. In general, however, antigens of the invention will be administered at a dose of between 0.1 and 100 μg of each antigen per dose, with a typical dosage volume of 0.5 ml. The dose is typically between 5 and 20 μg per antigen per dose.

The amount of CD1d ligand administered to the patient to induce an immune response may vary depending on the age and weight of a patient to whom the composition is administered but will typically contain between 1-100 μg/kg patient bodyweight. Surprisingly, it has been found that low doses of the CD1d ligand are sufficient to enhance the immune response to a co-administered antigen and promote long-term immunological memory to that antigen. The amount of CD1d ligand included in the compositions of the invention may therefore be less than 50 μg/kg patient bodyweight, less than 20 μg/kg, less than 10 μg/kg, less than 5 μg/kg, less than 4 μg/kg, or less than 3 μg/kg.

Vaccines according to the invention may either be prophylactic (i.e. to prevent infection) or therapeutic (i.e. to treat disease after infection), but will typically be prophylactic.

The invention provides a CD1d ligand and an antigen from group B streptococcus for use in medicine. The invention provides a CD1d ligand and an antigen from N. meningitidis serogroup B for use in medicine. The invention provides a CD1d ligand and an antigen from influenza virus selected from an influenza strain which is capable of or has the potential for causing a pandemic outbreak for use in medicine.

The invention also provides a method of raising an immune response in a patient, comprising administering to a patient a vaccine according to the invention. In particular, the invention provides a method of raising an immune response in a patient, comprising administering to a patient a CD1d ligand and an antigen from group B streptococcus. The invention provides a method of raising an immune response in a patient, comprising administering to a patient a CD1d ligand and an antigen from N. meningitidis serogroup B. The invention provides a method of raising an immune response in a patient comprising administering a CD1d ligand and an antigen from influenza virus selected from an influenza strain which is capable of or has the potential for causing a pandemic outbreak.

The antigen and CD1d ligand may be administered simultaneously, sequentially or separately. For example, the CD1d ligand may be administered to prime the mammal before administration of the antigen or after the administration of the antigen to boost the mammal's immune response to that conjugate. Where more than one antigen is being administered, the antigens may be administered simultaneously with the CD1d ligand being administered separately, simultaneously or sequentially to the mixture of antigens. The method of raising an immune response may comprise administering a first dose of an antigen and a CD1d ligand, and subsequently administering an optional second unadjuvanted dose of the antigen. The first dose of the antigen and CD1d ligand may be administered simultaneously, sequentially or separately.

The immune response is preferably a protective response and may comprise a humoral immune response and/or a cellular immune response. The patient may be an adult or a child. The patient may be aged 0-6 months, 6-12 months, 1-5 years, 5-15 years, 15-55 years or greater than 55 years. Preferably, the patient is a child.

The patient may be immunocompromised. The patient may have a disorder associated with lack of function of the immune system, and in particular a disorder associated with lack of function in CD4 T cell responses. Examples of such disorders include, but are not limited to, AIDS, ataxia-telangiectasia, DiGeorge syndrome, panhypogammaglobulinemia, Wiscott-Aldrich syndrome and complement deficiencies.

The invention provides the use of an antigen from group B streptococcus in the manufacture of a medicament for raising an immune response in a patient, wherein the medicament is administered with a CD1d ligand. The invention provides the use of a CD1d ligand in the manufacture of a medicament for raising an immune response in a patient, wherein the medicament is administered with an antigen from group B streptococcus. The invention provides the use an antigen from group B streptococcus and a CD1d ligand in the manufacture of a medicament for raising an immune response in a patient. The invention also provides the use of an antigen from group B streptococcus in the manufacture of a medicament for raising an immune response in a patient, where the patient has been pre-treated with a CD1d ligand. The invention provides the use of CD1d ligand in the manufacture of a medicament for raising an immune response in a patient, where the patient has been pre-treated with an antigen from group B streptococcus. The invention provides the use of an antigen from group B streptococcus in the manufacture of a medicament for raising an immune response in a patient, wherein said patient has been pre-treated with an antigen from group B streptococcus and a CD1d ligand.

The invention also provides the use of an antigen from N. meningitidis serogroup B in the manufacture of a medicament for raising an immune response in a patient, wherein the medicament is administered with a CD1d ligand. The invention also provides the use of a CD1d ligand in the manufacture of a medicament for raising an immune response in a patient, wherein the medicament is administered with a an antigen from N. meningitidis serogroup B. The invention also provides the use an antigen from group B streptococcus and a CD1d ligand in the manufacture of a medicament for raising an immune response in a patient. The invention also provides the use of an antigen from N. meningitidis serogroup B in the manufacture of a medicament for raising an immune response in a patient, where the patient has been pre-treated with a CD1d ligand. The invention further provides the use of CD1d ligand in the manufacture of a medicament for raising an immune response in a patient, where the patient has been pre-treated with an antigen from N. meningitidis serogroup B. The invention also provides the use of an antigen from N. meningitidis serogroup B in the manufacture of a medicament for raising an immune response in a patient, wherein said patient has been pre-treated with an antigen from N. meningitidis serogroup B and a CD1d ligand.

The invention also provides the use of an antigen from an influenza virus (as described above) in the manufacture of a medicament for raising an immune response in a patient, wherein the medicament is administered with a CD1d ligand. The invention also provides the use of a CD1d ligand in the manufacture of a medicament for raising an immune response in a patient, wherein the medicament is administered with an antigen from an influenza virus (as described above). The invention also provides the use an antigen from an influenza virus (as described above) and a CD1d ligand in the manufacture of a medicament for raising an immune response in a patient. The invention also provides the use of an antigen from influenza virus (as described above) in the manufacture of a medicament for raising an immune response in a patient, where the patient has been pre-treated with a CD1d ligand. The invention further provides the use of CD1d ligand in the manufacture of a medicament for raising an immune response in a patient, where the patient has been pre-treated with an antigen from an influenza virus (as described above). The invention also provides the use of an antigen from an influenza virus (as described above) in the manufacture of a medicament for raising an immune response in a patient, wherein said patient has been pre-treated with an antigen from an influenza virus (as described above) and a CD1d ligand.

The medicament is preferably an immunogenic composition (e.g. a vaccine). The medicament is preferably for the prevention and/or treatment of a disease caused by group B Streptococcus, Neisseria meningitidis (e.g. meningitis, septicaemia etc.), or by influenza virus.

Vaccines can be tested in standard animal models (e.g. see ref. 252).

The invention further provides a kit comprising: a group B streptococcal antigen and a CD1d ligand. The invention further provides a kit comprising an antigen from N. meningitidis serogroup B and a CD1d ligand. The invention further provides a kit comprising an antigen from influenza virus and a CD1d ligand. The antigen and ligand are preferably supplied as separate components of the kit such that they are suitable for separate administration e.g. into different limbs.

### DEFINITIONS

The term “comprising” encompasses “including” as well as “consisting” e.g. a composition “comprising” X may consist exclusively of X or may include something additional e.g. X+Y.

The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

### BRIEF DESCRIPTION OF THE DRAWINGS

FIG. 1 shows geometric Ig titers.
A) Serum titers of protein-specific antibodies in C57/BL6 mice immunised intramuscularly with α-GC and bacterial (TT, tetanus toxoid, or DT, diphtheria toxoid) or viral proteins (H3N2, the haemoagglutinin-neuroaminidase subunit from influenza strain A). Mice immunised with proteins and α-GC (closed boxes) displayed higher antibody titers than mice immunised with proteins alone (open boxes). X axis shows time in days.
B) Mice bearing iNKT cells (Ja18+/+) immunised with H3N2 and α-GC (closed boxes) show enhancement in serum titer of H3N2-specific antibodies compared to mice bearing iNKT cells immunised with H3N2 alone (open boxes). Mice lacking iNKT cells (Ja18−/−) immunised with H3N2 and α-GC (closed boxes) show no enhancement in serum titer of H3N2-specific antibodies compared to mice lacking iNKT cells immunised with H3N2 alone (open boxes). All immunisations were subcutaneous.

FIG. 2 shows geometric mean IgG titers.
A) α-GC is as potent as CFA, CpG, MF59 and Alum in the production of both IgG1 and IgG2a antibodies. Antigen was TT.
B) MHC-II−/− mice immunised subcutaneously with H3N2 mounted detectable antibody (IgG) titers whereas MHC-II−/− mice immunised subcutaneously with H3N2 alone or in alum did not.

FIG. 3: Comparison of α-GC and MF59 in a mouse model of influenza virus infection. All immunisations were intramuscular.
A) H1N1-specific IgG titers (geomean). Mice immunised with H1N1 and α-GC have antibody titers that are comparable to those of mice immunized with H1N1 and MF59 and that are significantly higher than titers found in mice immunized with protein vaccine alone.
B) Survival percentage vs. days after challenge. 80% of mice immunised with H1N1 and α-GC and 100% of mice immunised with H1N1 and MF59 are alive after challenge with influenza virus.

FIG. 4 shows H3N2 Ig titers (geomean). White boxes are adjuvant-free; shaded boxes have α-GC:
A) IgG1 and IgG2a response of WT, IL-4−/− and IFN-γR−/− mice to subcutaneous immunisation with H3N2 and α-GC (shown in black where present) or subcutaneous immunisation with H3N2 alone (shown in white where present). Immunisation of wild-type mice with H3N2 alone induced an IgG1 response (Th2), whereas immunisation with H3N2 and α-GC induced an IgG1 and IgG2a response (Th0). Immunisation of IL4−/− mice with H3N2 alone did not induce an IgG response, whereas immunisation with H3N2 and α-GC induced an IgG1 and IgG2a response (Th0). Immunisation of IFN-γR−/− mice with H3N2 alone induced an IgG1 response (Th2) and immunisation with H3N2 and α-GC induced a significantly higher IgG1 response (Th2). Dotted line shows the minimal dilution of sera tested.
B) Mice treated with the anti-CD40L monoclonal antibody before and during subcutaneous immunisation with H3N2 and α-GC display H3N2 antibody titers that are significantly lower than those observed in mice treated with control IgG.

FIG. 5:
A) Mice were primed at 0 and 2 weeks with H3N2 alone or H3N2 and α-GC. 30 weeks after first immunisation, both groups of mice were boosted with H3N2 protein alone. Figure shows H3N2-Ig titer (geomean) vs. time (weeks). Arrows show time of immunisations. Mice primed with two doses of H3N2 and α-GC and subsequently boosted with protein alone (closed boxes) displayed antibody titers significantly higher than mice primed and boosted with H3N2 alone (open boxes). Immunisations were subcutaneous.
B) Frequency of H3N2-antibody secreting cell precursors in mice primed according to FIG. 5A (H3N2-IgG ASC precursors per million B cells). Frequency of H3N2-antibody secreting cell precursors at week 30 was significantly higher in mice immunised twice with H3N2 and α-GC (shaded) than in mice immunised twice with H3N2 alone (white).

FIG. 6: H3N2-Ig titer (geomean) vs. time (weeks). Decay of H3N2-specific antibodies in mice lacking iNKT cells (Jaα 18−/−) and mice bearing iNKT cells (Jaα 18+/+) immunised twice subcutaneously with H3N2 alone. Antigen-specific antibodies decay faster in mice lacking iNKT cells (triangles) than in mice bearing iNKT cells (circles).

FIG. 7 shows frequency of ASC precursors (i.e. memory B cells) in C57BL/6 mice 6 weeks after the last (of two) immunization with Tetanus Toxoid+/− adjuvants, as number per million B cells. C57BL/6 mice were immunised intramuscularly on day 0 and day 14 with tetanus toxoid with no adjuvant, α-GC or alum. Mice immunised with tetanus toxoid and α-GC displayed significantly higher frequency of TT-specific memory B cells than mice immunised with tetanus toxoid alone, whole mice immunised with tetanus toxoid in alum did not. * indicates p<0.05 vs. antigen-free administration, and ** indicates p<0.01. *** indicates p<0.05 vs. TI, w/o adjuvant.

FIG. 8: Immunisation schedule used to assess whether α-GC is required to be present in all vaccine doses used at priming. 20 C57BL/6 female mice, aged 6-8 weeks, were divided into 4 groups of 5 mice. Group 1) was immunised with H3N2 in PBS at week 0 and H3N2+α-GC 2 weeks later. Group 2) were immunised with H3N2+α-GC at week 0 and H3N2 in PBS 2 weeks later. Group 3) were immunised with H3N2 in PBS at week 0 and week 2. Group 4) were immunised with H3N2 and α-GC at week 0 and 2 weeks later. 56 weeks after the initial immunisation, mice in all groups were challenged with 3 μg H3N2 in PBS and recall response was assessed at 58 weeks. All immunisations were intramuscular.

FIG. 9: Comparison of the H3N2-antibody response of the mice in group 3 of FIG. 8 (immunised twice with H3N2 in PBS) with: A) the responses in mice group 4 of FIG. 8 (immunised twice with H3N2 in α-GC); B) the response of mice in group 1 of FIG. 8 (immunisation with H3N2 in PBS and then with H3N2 in α-GC); and C) the responses in mice in group 2 of FIG. 8 (immunisation with H3N2 in α-GC and then with H3N2 in PBS). There were no differences in antibody half life.

FIG. 10: Comparison of the H3N2-antibody response observed following: A) immunisation twice with α-GC (group 4) versus α-GC in the second immunisation only (group 1); B) mice immunised twice with α-GC (group 4) versus α-GC in the first immunisation only (group 2); and C) immunisation with α-GC in the first immunisation only (group 2) versus immunisation with α-GC in the second immunisation only (group 1).

FIG. 11: Recall response of mice immunised as described in FIG. 8. Two weeks after a booster immunisation with H3N2 alone (given on week 56), mice primed with one or two doses of α-GC displayed higher recall responses the mice primed with 2 doses of H3N2 alone. Data are H3N21 g titers (geomean) at weeks 56 & 58.

FIG. 12: The frequencies of MenB-specific memory B cells in the spleen of the mice immunised as described in FIG. 13 were determined. Higher frequencies of MenB-specific memory B cells were found in the spleens of mice immunised with α-GC or MF59 compared to alum. Graph shows MenB-specific IgG memory B cells per million B lymphocytes. * & **: p<0.05 & p<0.01 vs. no adjuvant.

FIG. 13: Mice were immunised with a mixture of 3 MenB antigens, ΔG287nz-953, 936-741 and 961c (20 μg/dose, 5 μg/dose or 2.5 μg/dose each) admixed with 0.1 μg α-GC, 0.6 mg alum, 100 μl of MF59 or no adjuvant. A series of three immunisations was given on days 0, 21 and 35, and IgG titers to each antigen were assessed after each immunisation, up to day 105. Both α-GC and MF59 induced higher bactericidal antibody titers than alum.

FIG. 14: Comparison of the CD4 T cell response against recombinant MenB antigens in mice immunised intramuscularly on day 0 and 21 with: a) a combination vaccine containing 3 MenB antigens and α-GC; b) a combination vaccine containing 3 MenB antigens and Alum; or c) with a combination vaccine containing 3 MenB antigens alone. The CD4 T cell response was assessed two weeks after the second immunisation by incubating total splenocytes with the indicated amount of MenB recombinant proteins for 16 hours (the last 14 of which in the presence of Brefeldin A). The number CD4 T cells producing TNFa was determined by intracellular staining and FACS analysis). Mice immunised with combination of three MenB antigens and α-GC consistently displayed a higher CD4 response compared to mice immunised with combination of MenB antigens and alum or no adjuvant. As a positive control, response of all three groups of mice to polyclonal stimulation was tested. All three groups of mice showed the same response to polyclonal stimulation with an anti-CD3 antibody (IaCD3), as shown in insert of Figure. Y axis shows CD4 T cells producing TNFα as a percentage of all CD4+ T cells.

FIG. 15: Titers (geomean). Comparison of IgG, IgG1 and IgG2a titers in mice immunised with GBS antigens. Mice immunised with 1 μg GBS80 and α-GC showed significantly higher IgG1 and IgG2a titres than mice immunised with 1 μg GBS80 alone, while mice immunised with GBS80 in alum did not. Mice immunised with 20 μg GBS80 and α-GC showed equivalent IgG1 titers to mice immunised with 20 μg GBS80 and alum and greater IgG2a titers. *, ** p<0.05, p<0.01 vs GBS80 w/o adjuvant.

FIG. 16: Mice were immunised on day 0 and day 21 with one of three MenB antigens (DG287nz-953, 936-741 or 961c) or a mixture of all three antigens in combination with: a) α-GC; b) Alum; or c) no adjuvant. Levels of bactericidal antibodies against the MenB strains MC58, 2996, H44/76 and NZ98/254 were assessed two weeks after the second immunisation and two weeks after the third immunisation. Bactericidal antibodies were significantly higher when the combination vaccine was administered with α-GC as the adjuvant, compared to alum. All immunisations were intramuscular.

FIG. 17: Frequencies of memory B cells in the spleen of mother immunised with GBS80. Frequencies of plasma cells producing GBS80-specific antibodies were significantly higher in spleens from mothers immunised with GBS80 and α-GC than in the spleens of mothers immunised with GBS80 alone or with alum. Graph shows number of GBS80 IgG plasma cells per million B lymphocytes.

FIG. 18: Comparison of plasma cell frequencies of mothers immunised with GBS80 and α-GC and mothers immunised with GBS80 and alum. Plasma cell frequencies were significantly higher in mothers immunised with GBS80 and α-GC. Graph shows number of GBS80 IgG plasma cells per million B lymphocytes.

FIG. 19: Mice were immunised with a mixture of 3 MenB antigens, ΔG287nz-953, 936-741 and 961c (20 μg/dose each) alone, admixed with 0.1 μg α-GC, or admixed with 0.6 mg alum. A series of three immunisations was given on days 0, 21 and 35, and IgG titers to each antigen were assessed after each immunisation. α-GC was as effective as alum in enhancing the antibody response to all three of the MenB antigens in the combination vaccine. All immunisations were intramuscular.

### MODES FOR CARRYING OUT THE INVENTION

Additional information regarding modes for carrying out the invention can be found in reference 253.

### Example 1

### Invariant NKT Cells Help Protective Antibody Responses In Vivo and Contribute to Maintaining B-Cell Memory

### Summary

CD1d-restricted invariant natural killer T (iNKT) cells are innate-like lymphocytes that recognize glycolipid antigens such as α-galactosylceramide (α-GC). To investigate the effects of the innate immune system on adaptive immune responses in vivo, we assessed whether iNKT cells influenced critical features of the antibody response such as protection from infections and B cell-memory. We immunised mice with bacterial or viral proteins in combination with α-GC and found that mice immunised with proteins and α-GC develop antibody titers that are one to two logs higher than titers induced by proteins alone and, most important, they are more protected from infections such as Influenza. Mice that lack MHC class II do not produce antibodies when immunised with proteins and conventional adjuvants, however, immunisation of these mice with proteins and α-GC elicits detectable IgG specific for the protein, demonstrating that iNKT cells can partially replace the help to B cells by class-II restricted CD4+ T cells. Finally, we have found that mice immunised with proteins and α-GC have a frequency of protein-specific memory-B-cells that is higher than the frequency observed in mice immunised with the proteins alone. Moreover, mice lacking iNKT cells exhibit a decay of circulating antibody titers that is faster than the decay observed in wild type mice, suggesting an unexpected influence of iNKT cells on the lifespan of plasma cells. Altogether, these findings point to an important role of iNKT cells in the regulation of the antibody response and the maintenance of B cell memory in vivo.

### Results

Activation of iNKT Cells Enhances the Antibody Response to Protein Antigens In Vivo.

We have recently demonstrated that human iNKT cells can help B lymphocytes to proliferate and to produce immunoglobulins in vitro. To determine the in vivo relevance of this finding, we immunised C57/BL6 mice with bacterial (TT, tetanus toxoid, or DT, diphtheria toxoid) or viral proteins (H3N2, the haemoagglutinin-neuroaminidase subunit from influenza A strains) with or without the NKT specific glycolipid, α-GC, and assessed serum titers of protein-specific antibodies at various time points. FIG. 1A shows that with all antigens, mice immunised with proteins and α-GC (closed boxes) displayed antibody titers one to two logs higher than titers of mice immunized with proteins alone (open boxes). Similar results were obtained in BALB/c, CD1 and C3H/HeJ mice (data not shown).

To prove that the adjuvant activity of α-GC was due to activation of iNKT cells, we immunised mice bearing (Ja18+/+ and Ja18+/−) or lacking (Ja18−/−) iNKT cells with the Flu proteins H3N2 with or without α-GC. As shown in FIG. 1B, all mice immunised with H3N2 alone (open boxes) developed comparable antibody responses, regardless of the presence of iNKT cells. However, FIG. 1B shows that when immunization is done with H3N2 and α-GC (closed boxes), mice bearing iNKT cells show a significant enhancement in the serum titer of H3N2-specific antibodies, whereas mice lacking iNKT fail to do so. These results were strengthened by the finding (data not shown) that α-GC did not display adjuvant activity in mice lacking CD1d (CD1−/−), the restriction element that presents α-GC to the T cell receptor of iNKT cells.

To compare α-GC activity to that of more conventional adjuvants, mice were immunised with increasing doses of TT given alone, with α-GC or with an optimal dose of one of the following adjuvants: CFA (one of the strongest adjuvant that is used in mice [254]), CpG (a strong Th0/Th1 immunostimulator that is currently tested in man 255), MF59 and Alum (two adjuvants licensed for human use [256, 257], both considered Th0/Th2 inducers). As shown in FIG. 2A, α-GC is overall as potent as the above benchmark adjuvants in helping the production of both IgG1 and IgG2a antibodies.

Finally, we assessed whether antibody responses to proteins antigens could develop with the help of iNKT lymphocytes in the absence of conventional CD4+ T cell help, a situation where conventional adjuvants fail to provide help. Thus, two groups of C57BL/6 mice that lack MHC class II molecules (MHC-II−/−) were immunized twice with H3N2, given alone or with α-GC or with Alum. As expected, MHC-II−/− mice immunized with H3N2 alone or in Alum did not show any antigen specific antibodies (FIG. 2B). Instead, MHC-II−/− mice immunized with H3N2 and α-GC mounted detectable antibody (IgG) titers.

Altogether, these results demonstrate that iNKT cells activated in vivo by α-GC potentiate antibody responses to protein antigens in a manner comparable to that of conventional adjuvants. At a variance with these adjuvants, α-GC does not require MHC-class II-restricted CD4 T lymphocytes to generate an antibody response.

iNKT Cells Help Immunity

Having demonstrated that α-GC enhances the antibody response to pathogen proteins, we addressed the quality of the response and asked whether these antibodies were capable of protecting from infections. To this end, we compared the adjuvant effect of α-GC to that of MF59 (an adjuvant licensed for human use with Flu vaccines) in a mouse model of influenza virus infection. Adult C57BL/6 mice were immunized, at day 0 and 15, with the H1N1 proteins (from the human influenza virus A/NewCaledonia/20/99) alone, with α-GC or with MF59. Two weeks after the last immunization, mice were challenged with a 90% lethal dose (LD) of the mouse-adapted A/WS/33 Flu virus, and their survival was followed up for two weeks. As shown in FIG. 3A, one day before challenge, mice immunized with H1N1 and α-GC have antibody titers that are comparable to those of mice immunized with H1N1 and MF59 and that are significantly higher than titers found in mice immunized with the protein vaccine alone. Moreover, FIG. 3B shows that two weeks after challenge, 80% of mice immunized with H1N1 and α-GC, and 100% of mice immunised with proteins and MF59 were alive, whereas only 10% of mice that were immunized with the vaccine based on the proteins alone were still alive at the end of follow up.

Altogether, from these results we conclude that α-GC-dependent iNKT cell activation can enhance the efficacy of vaccines against infectious diseases.

Mechanism of iNKT Cells Help to B Cells

We next examined the mechanisms driving the iNKT cell help to B cells in vivo.

First, to investigate the role of cytokines, we assessed the adjuvant effect of α-GC both in C57BL/6 mice and in congenic mice lacking the cytokine IL-4 or the receptor for IFN-γ(IFN-γR). FIG. 4A (left panel) shows that in wild type mice, immunisation with the flu proteins H3N2 alone (shown in white) induced a Th2 response as indicated by the presence of IgG1 and the absence of IgG2a, whereas immunisation with protein and α-GC elicited a balanced Th0 response, as demonstrated by the presence of both IgG1 and IgG2a (shown in black). The mid-panel of FIG. 4A shows that mice lacking IL-4 did not have any antibody response when immunised with the protein alone, whereas they mounted a balanced Th0 response when immunised with protein and α-GC (shown in black). Finally, mice lacking IFN-γ receptor (FIG. 4A, right panel) display a Th2 response (IgG1 antibodies) when immunised with the protein alone (shown in white). Although IgG1 titers increase significantly in mice immunised with protein and α-GC (shown in black), there is no increase in IgG2a antibodies above background levels. Altogether these findings demonstrate that IL-4 is individually dispensable for the α-GC-dependent iNKT cell to help B lymphocytes, whereas IFN-γ is essential for a balanced (Th0) helper effect of iNKT cells.

Second, we asked whether CD40/CD40L interactions were required for the α-GC-dependent iNKT cell help in vivo. We therefore assessed antibody responses to H3N2 in mice treated with saturating amounts of a neutralizing anti-CD40L mAb. As shown in FIG. 4B, following immunisation with H3N2 and α-GC, mice treated with the anti-CD40L mAb displayed H3N2 antibody titers that are significantly lower than those observed in mice treated with control IgG.

### α-GC Enhances Recall Antibody Responses and Contribute to Maintain B Cell Memory.

A key feature of the adaptive immune system is the ability to mount a quicker “recall” response to an antigen it has encountered previously. To assess whether the adjuvant effect of α-GC influenced recall antibody responses, mice were immunised twice, at week 0 and 2, with H3N2 alone or with α-GC. A third (recall) immunisation with H3N2 alone was then given to all mice at week 30. FIG. 5A shows that, in agreement with data reported in FIG. 1, after the first two doses, mice immunized with H3N2 and α-GC displayed antibody titers significantly higher than titers from mice receiving H3N2 alone. H3N2-specific antibodies decayed over time reaching background levels in both groups at about week 30, when all mice were boosted with a third immunisation with H3N2 alone. Two weeks later, we assessed antibody responses and found that mice that were given protein and α-GC in the first two immunizations displayed post-third antibody titers significantly higher than those of mice that were immunized in all three immunisations with the protein alone (FIG. 5A). Consistent with these results, in a parallel experiment we have found that the frequency of H3N2-antibody secreting cell (ASC) precursors detected at week 30 ((just before the third immunisation) in the spleen of mice immunized twice with H3N2 and α-GC was significantly higher than the frequency observed in the spleen of mice immunized twice with H3N2 alone (FIG. 5B).

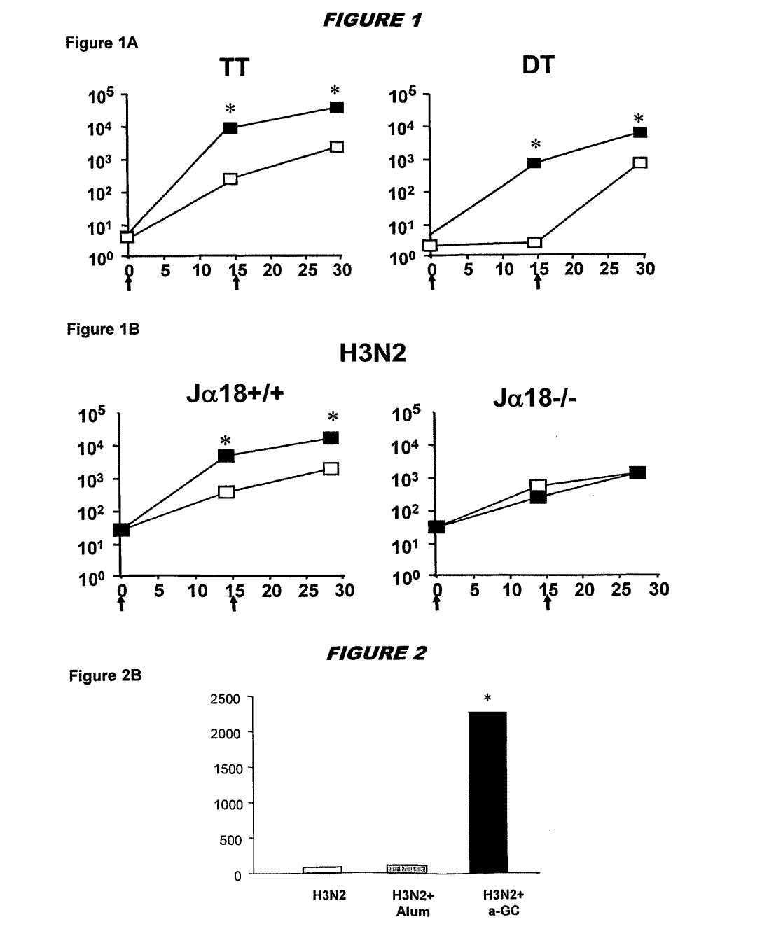

To further investigate the role of iNKT cells in the regulation of B cell memory, we assessed the persistence of antigen-specific antibodies induced by a protein (H3N2) alone in the sera of mice bearing (Ja18+/+ and Ja18+/−), or lacking (Ja18−/−) iNKT cells. In all groups of mice, titers of antigen specific antibodies peaked at comparable levels two weeks after the second immunisation. However, FIG. 6 shows that while in mice bearing iNKT cells antigen-specific antibodies decayed with a similar slow rate, the antibody titer decay in mice lacking iNKT cells was significantly faster. As none of these mice received α-GC, we conclude that some level of iNKT “spontaneous” activity can influence the half-life of circulating antibodies.

Altogether, these findings demonstrate that iNKT cell activation results in a higher antibody response to a recall immunisation and that this is due to an increased expansion of the antigen-specific memory B cell pool. Moreover, iNKT spontaneous activity in vivo appears to play a homeostatic role in maintaining circulating antibody levels.

### Example 2

### Priming with α-GalCer in the Absence of a Boost Significantly Enhances Antibody Response

As discussed above, the frequency of H3N2-antibody secreting cell (ASC) precursors (i.e. memory B cells) detected at week 30 (just before the third immunisation) in the spleen of mice immunized twice with H3N2 and α-GC was significantly higher than the frequency observed in the spleen of mice immunized twice with H3N2 alone (FIG. 5B).

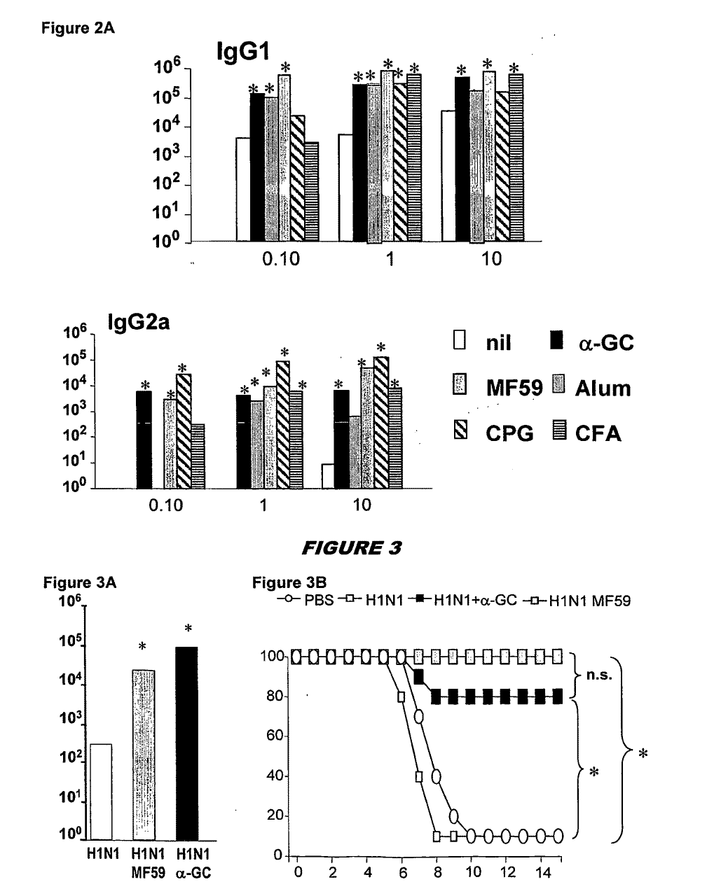

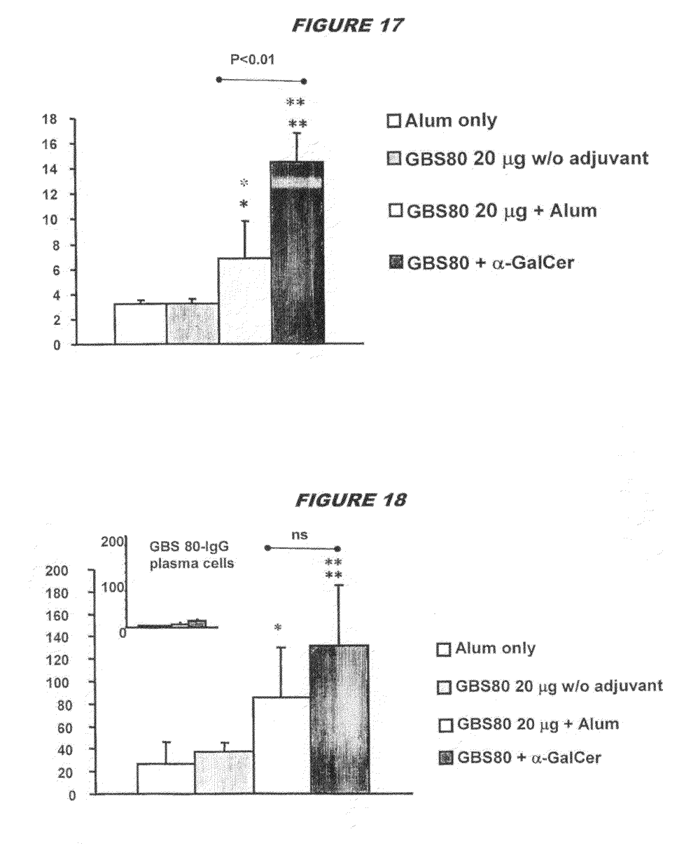

Similar results were obtained in experiments conducted in mice immunized with tetanus toxoid (FIGS. 7 and 18). FIG. 7 shows the frequency of ASC precursors in C57BL/6 mice 6 weeks after the last of two immunizations on day 0 and day 14 with tetanus toxoid with no adjuvant, with α-GC adjuvant or with alum adjuvant. The use of α-GC as an adjuvant significantly enhanced the frequency of ASC precursors compared to the use of an alum adjuvant. Likewise, FIG. 18 shows the frequency of ASC precursors in CD1 mice was significantly higher three month after the last of two immunisations with GBS80 and α-GC compared with immunisations with GBS80 and alum.

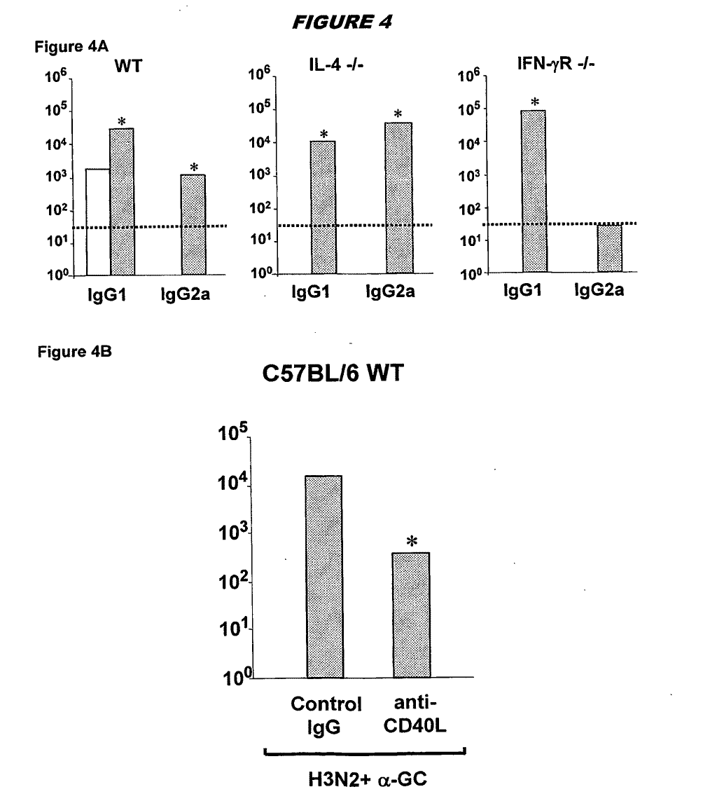

The ability of α-GC to significantly enhance the frequency of memory B cells compared to alum when administered as an adjuvant in a series of two immunisations suggested that α-GC might be capable of inducing an increase in the specific memory B cells when used as an adjuvant in a single immunisation. An experiment was therefore conducted to assess the effect of a single dose of α-GC and H3N2 antigen on the specific B cell memory pool (FIGS. 8-11).

FIG. 8 shows the immunisation schedule used in the experiment. 20 C57BL/6 female mice, aged 6-8 weeks, were divided into 4 groups of 5 mice. Group 1) was immunised with H3N2 in PBS at week 0 and H3N2+α-GC 2 weeks later. Group 2) were immunised with H3N2+α-GC at week 0 and H3N2 in PBS 2 weeks later. Group 3) were immunised with H3N2 in PBS at week 0 and week 2. Group 4) were immunised with H3N2 and α-GC at week 0 and 2 weeks later. 56 weeks after the initial immunisation, mice in all groups were challenged with 3 μg H3N2 in PBS. All immunisations were intramuscular.

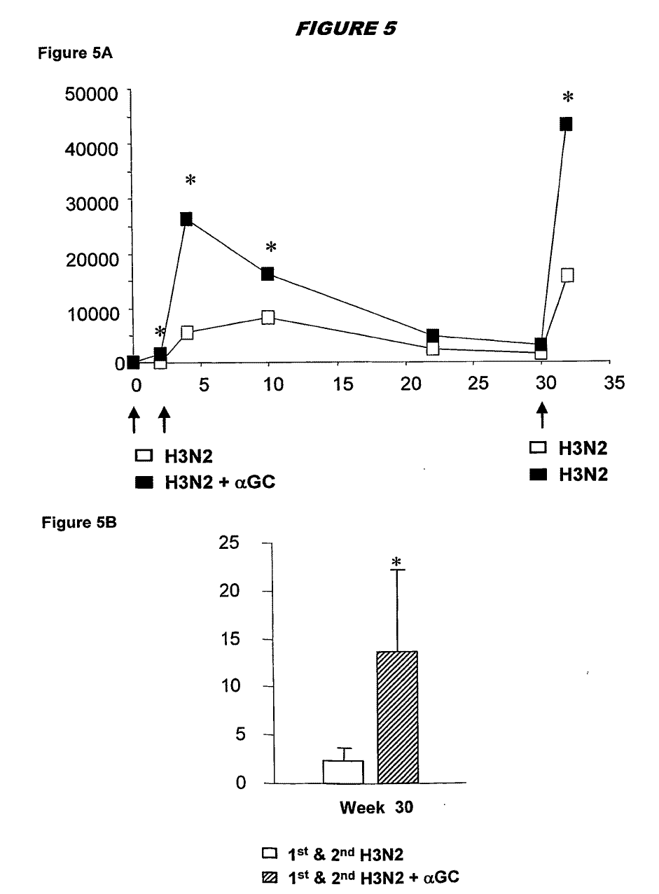

FIG. 9 compares the H3N2-antibody response of the mice in group 3 (immunised with H3N2 in PBS) with the responses in the mice immunised with α-GC in both immunisations (panel A), α-GC in the first immunisation only (panel B) and α-GC in the second immunisation only (panel C). α-GC was found to enhance the antibody response even when given only in the first or second immunisation. No differences in antibody half-life were observed between the four groups.

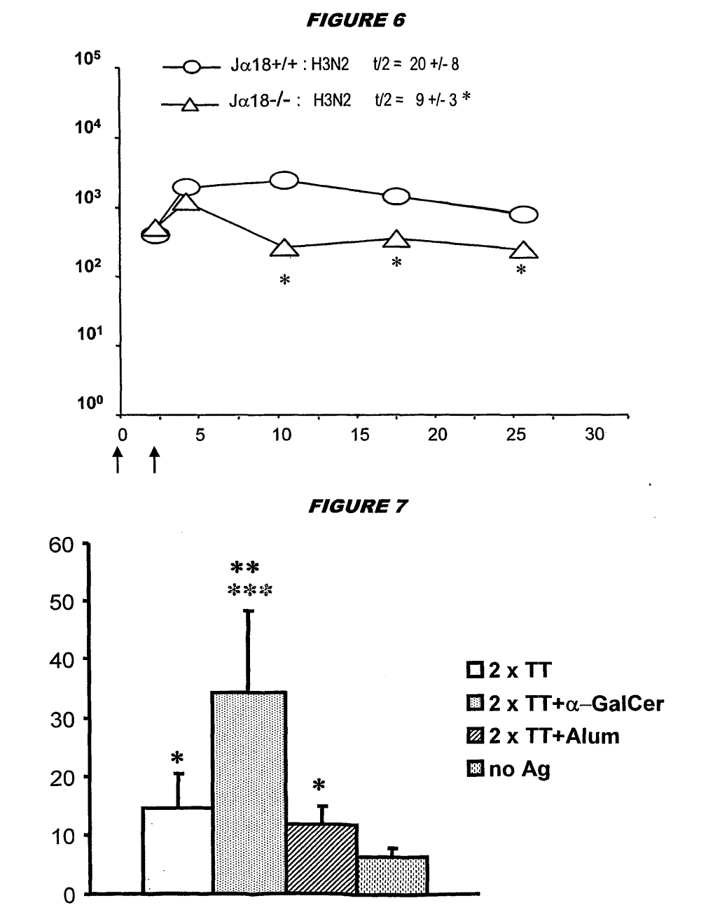

FIG. 10 provides pairwise comparisons of the antibody response observed following: A) immunisation twice with α-GC versus α-GC in the second immunisation only; B) mice immunised twice with α-GC vs α-GC in the first immunisation only; and C) immunisation with α-GC C in the first immunisation only versus immunisation with α-GC in the second immunisation only. Maximal efficacy was observed when α-GC was given in the first vaccine dose. Supplying α-GC in the first vaccine dose produced a high antibody response to supplying it in the second vaccine dose (FIG. 10C) and the antibody response when α-GC was supplied in the first vaccine dose was similar to the response obtained when α-GC was supplied in both vaccine doses (FIG. 10B).

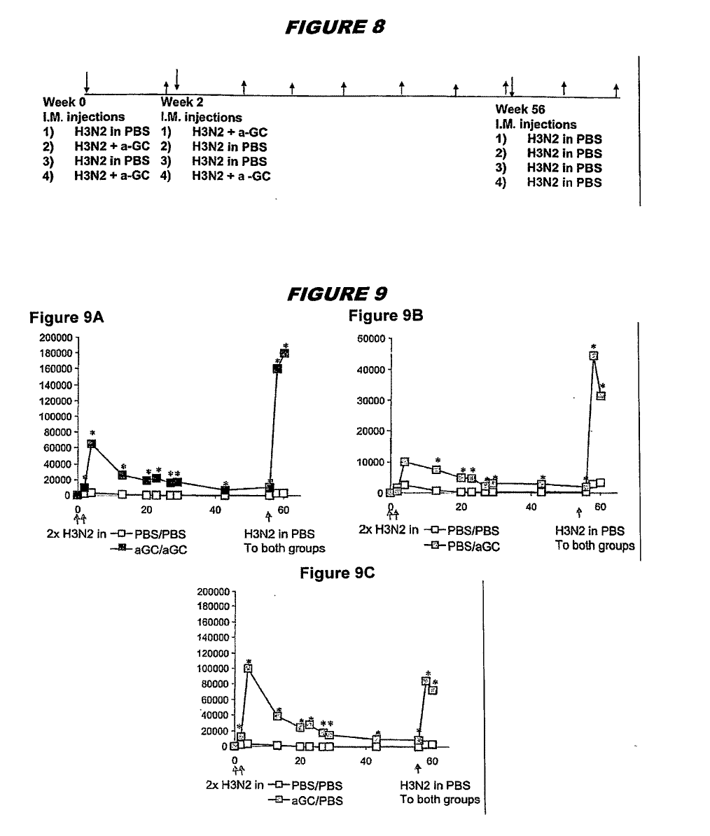

FIG. 11 confirms that mice primed with α-GC display a high recall response to vaccination, even if the α-GC is only included in the first or second dose of two priming injections. These results suggest that the inclusion of α-GC as an adjuvant in vaccine compositions may reduce the number of priming immunisations required to achieve long-term immunological memory and reduce the frequency and number of booster immunisations.

### Example 3

### α-GC Enhances Protective Antibody Responses in a Mouse Model of Neonatal Sepsis Induced by Streptococcus agalatiae

α-GC was tested for its ability to enhance protective antibody responses in a mouse model of neonatal sepsis induced by Streptococcus agalactiae infection.

Female mice were divided into 3 groups. Group 1 was primed on day 0 with 20 μg GBS80 in the absence of adjuvant and boosted on day 21 with the same composition. Mice were mated on day 23 and bled on days 43-36 to allow assessment of GBS80-IgG titers prior to delivery of offspring on day 50-53. Offspring were challenged with a 90% lethal dose of S. agalactiae 0-48 hours from birth. 3 months after booster dose, mothers were sacrificed, spleens were removed and GBS80-IgG plasma cell precursor frequencies were assessed. The same immunisation schedule was followed for groups 2 and 3 except that mice in group 2 were primed and boosted with GBS80 and alum and mice in group 3) were primed and boosted with GBS80 and 0.1 μg α-GC.

Results were as follows:

| | Mothers' | | |
|---|---|---|---|
| | GBS80-IgG | Dead | % |
| | titer | Pups/Total | Offspring |
| Group | (geomean) | Pups | Survival |
| GBS80 | <50 | 40/40 | 0 |
| GBS80/Alum | 1,877 ** | 28/40 | 30 |
| GBS80/α-GC | 15,546 ** | 12/39 | 70 § |

** p < 0.01 vs GBS-80;
§ p < 0.001 vs GBS-80 in Alum

Thus the use of α-GC as an adjuvant induced a GBS80-IgG response in the mothers which was 8-fold higher than the IgG response induced by alum. The higher antibody response in the mother resulted in enhanced protection of their offspring from GBS infection. 70% of offspring from mother immunised with GBS80 and α-GC survived challenged with S. galactiae compared with just 30% of offspring of mothers immunised with GBS80 and alum.

The experiment was repeated with mice being immunised with either 20 μg or 1 μg of GBS80. All mice were primed on day 0, boosted on day 20, mated on day 34, and bled on day 48 before delivery of offspring on days 54-58. Offspring were immediately challenged with a 90% lethal dose of S. agalactiae and survival was assessed at 48 hours. Mothers were sacrificed 3 months after boosting and spleens were removed for assessment of GBS80-IgG plasma cell precursor frequencies. The mice were immunised with: 1 μg GBS80 with alum, α-GC or no adjuvant; 20 μg GBS80 with alum, α-GC or no adjuvant; or with PBS or alum adjuvant alone.

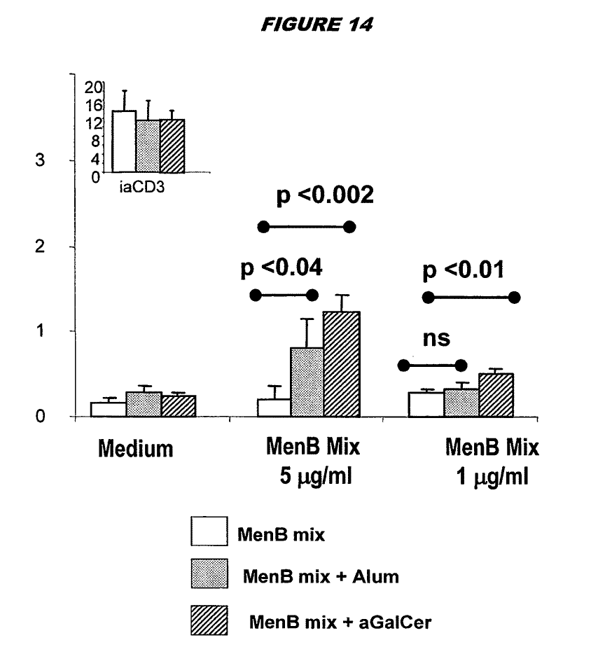

As shown in FIG. 15, mice immunised with 1 μg GBS80 and α-GC showed significantly higher IgG1 and IgG2a titres than mice immunised with 1 μg GBS80 and alum. Mice immunised with 20 μg GBS80 and α-GC showed equivalent IgG1 titers to mice immunised with 20 μg GBS80 and alum and greater IgG2a titers. Results were as follows:

| dead | total | % survival | Mother immunized with |
|---|---|---|---|
| 39.0 | 39.0 | 0.0 | PBS/Alum |
| 30.0 | 30.0 | 0.0 | GBS 80 1 mg |
| 21.0 | 39.0 | 46.2 | GBS 80/Alum 1 mg |
| 27.0 | 40.0 | 32.5 | a-Gal Cer GBS 80 1 mg |
| 39.0 | 40.0 | 2.5 | GBS 80 20 mg |
| 12.0 | 30.0 | 60.0* | GBS 80/Alum 20 mg |
| 21.0 | 40.0 | 47.5* | a-Gal Cer GBS 80 20 mg |

One-tail Fischer's Test:
*p < 0.05 vs GBS80 w/o adjuvant

Thus the % survival in mice immunised with GBS80 and α-GC was equivalent to survival in mice immunised with GBS80 and alum.

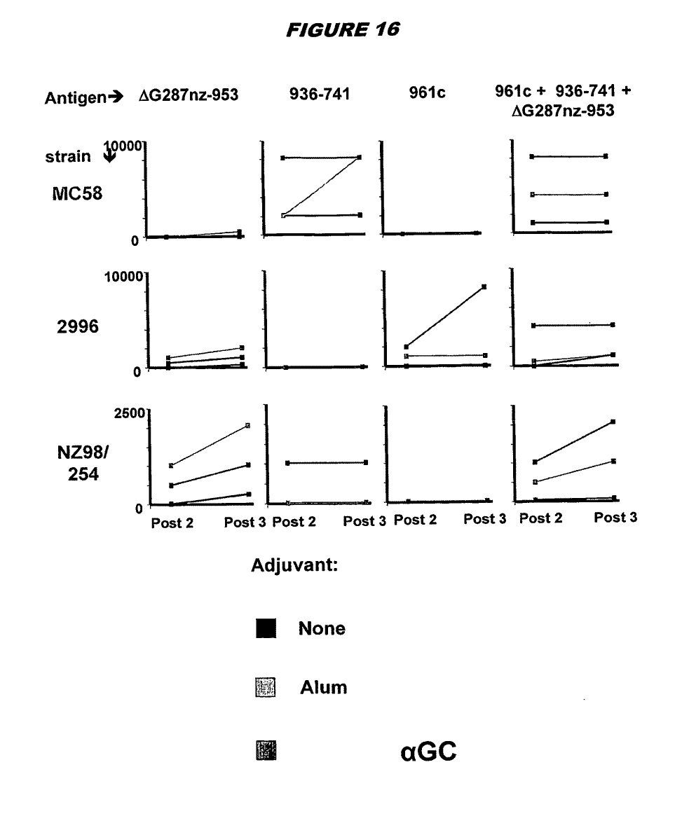

The spleens of mothers immunised with GBS80 and α-GC also contained significantly higher frequencies of GBS80 IgG plasma cells and memory B cells (FIGS. 17 and 18, respectively). frequencies of GBS80-specific plasma cells and memory b cells were determined by assessing the presence of GBS80-specific antibodies in 10-days supernatants from limiting dilution cultures of splenocytes incubated in medium alone or in the presence of CpG and IL-2, as described in ref. 258.

In a further experiment, pregnant CD1 female mice were immunised with PBS, GBS80, GBS80+Alum, or GBS80+α-GC. Sera taken one week before delivery from each group of immunised CD1 females, or from naïve CD1 females, were pooled and injected subcutaneously (3 μl/dose in a final volume of 20 μl) to 24 hours old neonates born from näive CD1 mothers. After 3 hours all neonates were challenged intraperitoneally with 1 LD₉₀ of live Streptococcus agalactiae. Pups' survival was followed up for 2 days. Pups immunised with sera from mothers immunised with GBS80 all died (28 pups out of 28) and only 1 of the 27 pups immunised with sera from mothers immunised with PBS survived. The presence of adjuvants (alum or α-GC) improved survival with immunisation with α-GC being more effective than alum in increasing survival. Survival of pups immunised with sera from mothers immunised with GBS+α-GC was 165% greater than survival of pups immunised with sera from mothers immunised with GBS+alum.

These data demonstrate that α-GC is surprisingly significantly more effective than alum in inducing a protective immune response to S. agalactiae.

### Example 4

### α-GC Enhances Antibody Response to Combination Vaccine Containing Several Protein Antigens from N. meningitidis Serogroup B

The ability of α-GC to act as an adjuvant for combinations of antigens from N. meningitidis serogroup B (MenB) antigens was assessed.

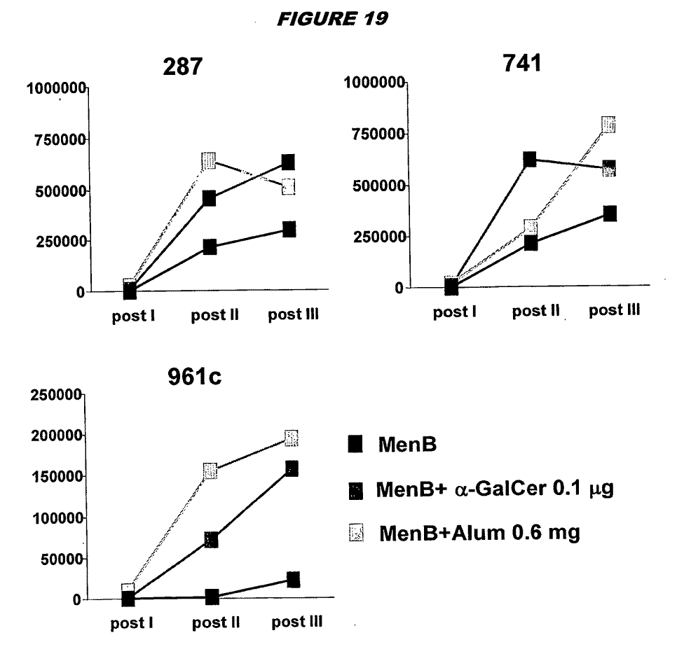

Mice were immunised with a mixture of 3 MenB antigens, ΔG287nz-953, 936-741 and 961c (20 μg/dose each) admixed with 0.1 μg α-GC, or admixed with 0.6 mg alum. A series of three immunisations was given on days 0, 21 and 35, and IgG titers to each antigen were assessed after each immunisation. As shown in FIG. 19, α-GC was as effective as alum in enhancing the antibody response to all three of the MenB antigens in the combination vaccine.

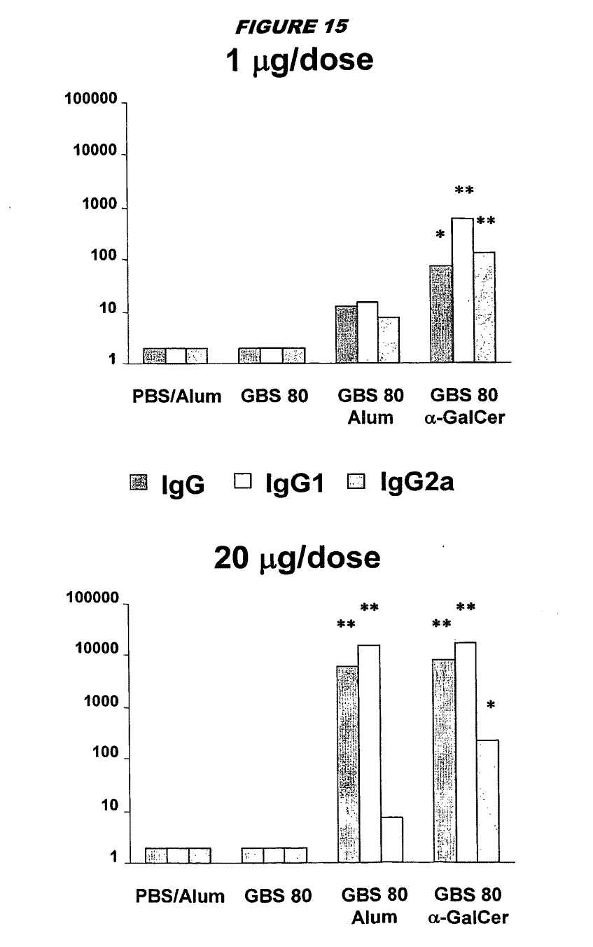

α-GC also enhanced the bactericidal response to these antigens. FIG. 16 compares the bactericidal antibody responses to MenB strains MC58, 2996, H44/76 and NZ98/254 in serum samples from mice immunised with a mixture of the 3 MenB antigens ΔG287nz-953, 936-741 and 961c or with each of these antigens individually in combination with alum, α-GC or no adjuvant. Bactericidal responses to immunisation with MenB antigens and α-GC was consistently higher than response to immunisation with MenB antigens and alum.

A second experiment was conducted to assess the ex vivo CD4 T cell response to MenB antigens. Groups of 6 CD1 female mice were immunised twice with the mix of MenB antigens, formulated in PBS, alum or α-GC. 10 days after the 2nd immunisation, 3 mice/group were sacrificed and their spleens were removed. Whole splenocyte suspensions from individual mice were cultured with the MenB antigens for 16 hours, the last 12 of which were in the presence of brefeldin A to allow intracellular accumulation of cytokines. Stimulated splenocytes were fixed, permeabilized and stained with anti-CD3, anti-Cd4, anti-CD69, anti-IFNg and anti-TNFa monoclonal antibodies. Percentages of CD3+CD4+CD69+cytokine+ cells in the total CD4+ cell population were determined by FACS analysis.

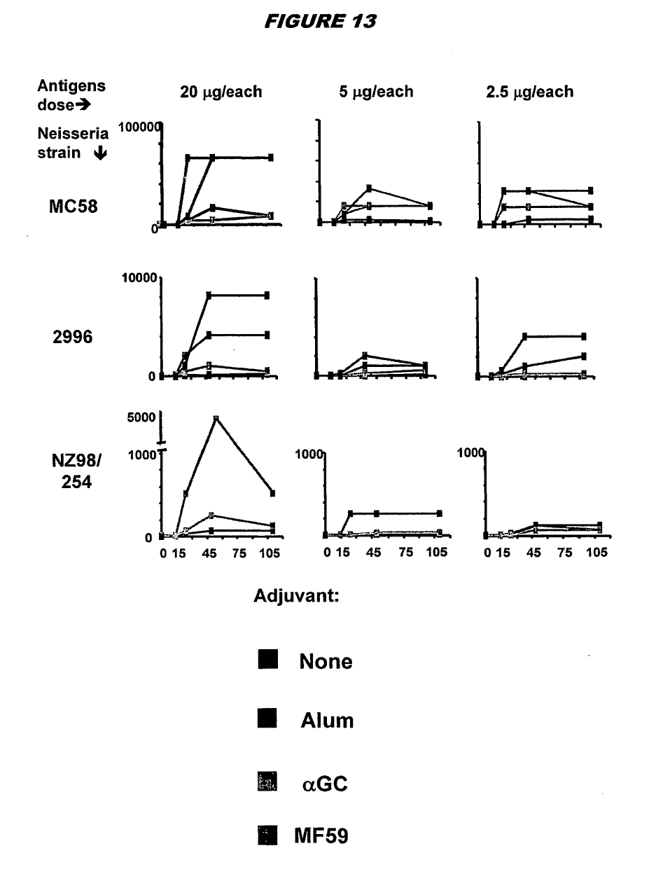

The results are shown in FIG. 14. α-GC was found to be more effective than alum in expanding CD4 T cells producing TNFα in response to MenB antigens, demonstrating that α-GC is able to induce a cell-mediated immune response to MenB antigens that is at least equivalent to alum. As a positive control, response of all three groups of mice to polyclonal stimulation was tested. All three groups of mice showed the same response to polyclonal stimulation with an anti-CD3 antibody (IaCD3), as shown in FIG. 14 insert.

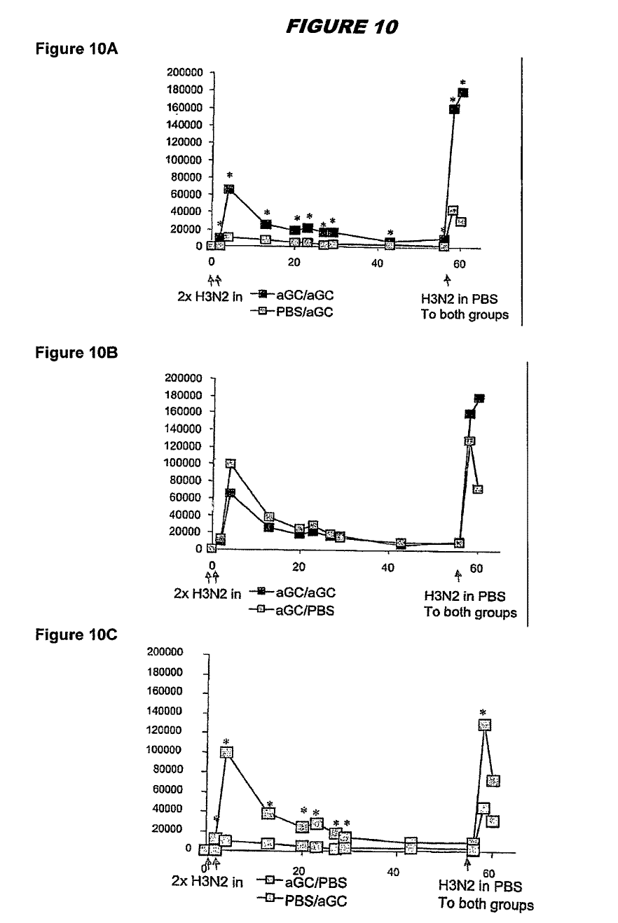

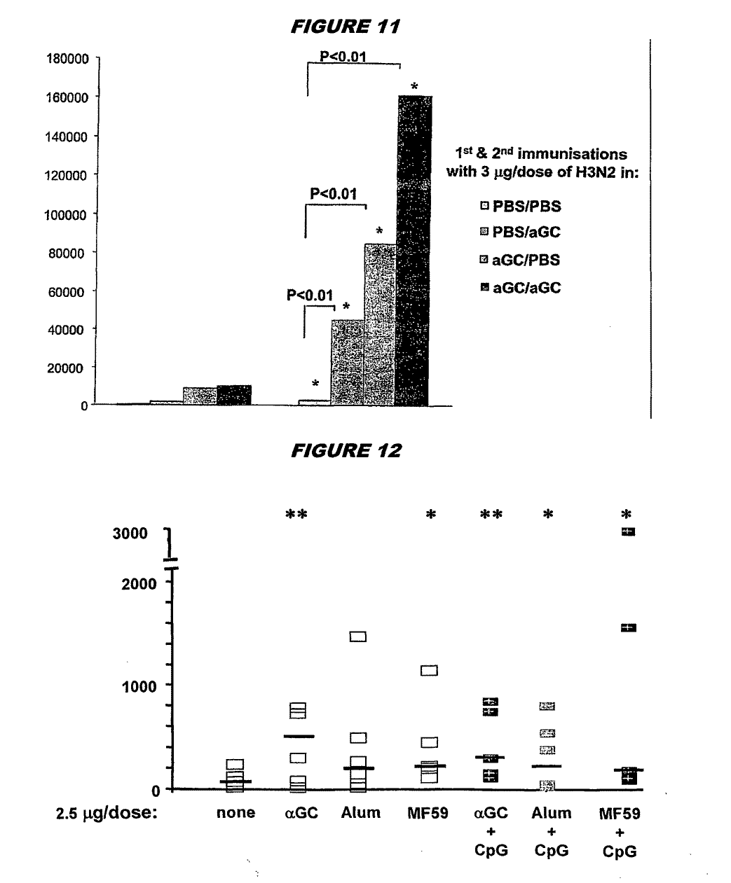

An additional experiment was conducted to assess the ability of α-GC to act as an adjuvant for the same three MenB antigens compared to alum or MF59. Mice were immunised with a mixture of 3 MenB antigens, ΔG287nz-953, 936-741 and 961c (20 μg/dose, 5 μg/dose or 2.5 μg/dose each) admixed with 0.1 μg α-GC, 0.6 mg alum, 100 μl of MF59 or no adjuvant. A series of three immunisations was given on days 0, 21 and 35, and IgG titers to each antigen were assessed after each immunisation. As shown in FIG. 13, both α-GC and MF59 induced higher bactericidal antibody titers than alum. The frequencies of MenB-specific memory B cells in the spleen of the immunised mice were also determined. As shown in FIG. 12, higher frequencies of MenB-specific memory B cells were found in the spleens of mice immunised with α-GC or MF59 compared to alum. These data show that α-GC is more effective than alum and as effective as MF59 in inducing a bactericidal immune response against MenB antigens and in inducing Men-B specific memory B cells required for long-term immunological memory.

It will understood that the invention has been described by way of example only and modification of detail may be made without departing from the spirit and scope of the invention.

### REFERENCES

[1] Natori et al, Tetrahedron, 1994, 50: 2771-2783
[2] EP-A-1018548
[3] Galli et al, Vaccine, 2003, 21: S2148-S2154
[4] Galli et al, J. Exp. Med., 2003, 197: 1051-1057
[5] WO03/009812
[6] Gonzalez-Aseguinolaza et al, J. Exp Med, 2002, 5: 617-624
[7] Huang et al., 2003, Abstract 396, 10th Conference on retroviruses and opportunistic infection
[8] Ko et al, J Immunol., 2005, 175: 3309-3317
[9] De Libero et al, Nature Reviews Immunology, 2005, 5: 485-496
[10] U.S. Pat. No. 5,936,076
[11] WO03/105769
[12] Oki et al, J. Clin. Investig., 113: 1631-1640
[13] US2005/0192248
[14] Yang et al, Angew. Chem. Int. Ed., 2004, 43: 3818-3822
[15] WO2005/102049
[16] Goff et al, J. Am. Chem., Soc., 2004, 126: 13602-13603
[17] Ndonye et al, J. Org. Chem., 2005, 70: 10260-10270
[18] Wu et al, PNAS, 2005, 102(5): 1351-1356
[19] Mattner et al, Nature, 2005, 434: 525-529
[20] Kinjo et al, Nature, 2006, 7(9): 978-986
[21] WO2005/033148.
[22] WO03/007985
[23] Wessels et al. (1990) J Clin Invest 86:1428-33.
[24] Wessels et al. (1989) Infect Immun 57:1089-94.
[25] International patent application PCT/IB03/01436.
[26] Ravenscroft et al. (1999) Vaccine 17:2802-2816.
[27] Costantino et al. (1999) Vaccine 17:1251-1263.
[28] Anonymous (January 2002) Research Disclosure, 453077.
[29] Anderson (1983) Infect Immun 39(1):233-238.
[30] Anderson et al. (1985) J Clin Invest 76(1):52-59.
[31] EP-A-0372501.
[32] EP-A-0378881.
[33] EP-A-0427347.
[34] WO93/17712
[35] WO94/03208.
[36] WO98/58668.
[37] EP-A-0471177.
[38] WO91/01146
[39] Falugi et al. (2001)Eur J Immunol 31:3816-3824.
[40] Baraldo et al, (2004) Infect Immun. 72:4884-7
[41] EP-A-0594610.
[42] WO00/56360.
[43] WO02/091998.
[44] Kuo et al. (1995) Infect Immun 63:2706-13.
[45] WO01/72337
[46] WO00/61761.
[47] WO99/42130.
[48] WO2004/011027.
[49] WO96/40242.
[50] Lees et al. (1996) Vaccine 14:190-198.
[51] WO95/08348.
[52] WO98/42721
[53] U.S. Pat. No. 4,882,317
[54] U.S. Pat. No. 4,695,624
[55] Mol. Immunol., 1985, 22, 907-919
[56] EP-A-0208375
[57] WO00/10599
[58] Gever et al., Med. Microbiol. Immunol, 165: 171-288 (1979).
[59] U.S. Pat. No. 4,057,685.
[60] U.S. Pat. Nos. 4,673,574; 4,761,283; 4,808,700.
[61] U.S. Pat. No. 4,459,286.
[62] U.S. Pat. No. 4,965,338
[63] U.S. Pat. No. 4,663,160.
[64] U.S. Pat. No. 4,761,283
[65] U.S. Pat. No. 4,356,170
[66] Lei et al. (2000) Dev Biol (Basel) 103:259-264.
[67] WO00/38711; U.S. Pat. No. 6,146,902.
[68] WO2006/027685.
[69] WO99/24578.
[70] WO99/36544.
[71] WO99/57280.
[72] WO00/22430.
[73] Tettelin et al. (2000) Science 287:1809-1815.
[74] WO96/29412.
[75] Pizza et al. (2000) Science 287:1816-1820.
[76] WO02/22167.
[77] Bell (2000) Pediatr Infect Dis J 19:1187-1188.
[78] Iwarson (1995)APMIS 103:321-326.
[79] Gerlich et al. (1990) Vaccine 8 Suppl:S63-68 & 79-80.
[80] Hsu et al. (999) Clin Liver Dis 3:901-915.
[81] WO03/002065
[82] WO01/37869
[83] WO04/005473
[84] Gustafsson et al. (1996) N. Engl. J. Med. 334:349-355.
[85] Rappuoli et al. (1991) TIBTECH 9:232-238.
[86] Vaccines (2004) eds. Plotkin & Orenstein. ISBN 0-7216-9688-0.
[87] WO02/02606.
[88] Kalman et al. (1999) Nature Genetics 21:385-389.
[89] Read et al. (2000) Nucleic Acids Res 28:1397-406.
[90] Shirai et al. (2000) J. Infect. Dis. 181 (Suppl 3):S524-S527.
[91] WO99/27105.
[92] WO00/27994.
[93] WO00/37494.
[94] WO99/28475.
[95] Ross et al. (2001) Vaccine 19:4135-4142.
[96] Sutter et al. (2000) Pediatr Clin North Am 47:287-308.
[97] Zimmerman & Spann (1999) Am Fam Physician 59:113-118, 125-126.
[98] Dreesen (1997) Vaccine 15 Suppl:S2-6.
[99] MMWR Morb Mortal Wkly Rep 1998 Jan. 16; 47(1):12, 19.
[100] Covacci & Rappuoli (2000) J. Exp. Med. 19:587-592.
[101] WO93/18150.
[102] Covacci et al. (1993) Proc. Natl. Acad. Sci. USA 90: 5791-5795.
[103] Tummuru et al. (1994) Infect. Immun. 61:1799-1809.
[104] Marchetti et al. (1998) Vaccine 16:33-37.
[105] Telford et al. (1994) J. Exp. Med. 179:1653-1658.
[106] Evans et al. (1995) Gene 153:123-127.
[107] WO96/01272 & WO96/01273, especially SEQ ID NO:6.
[108] WO97/25429.
[109] WO98/04702.
[110] McMichael (2000) Vaccine 19 Suppl 1:S101-107.
[111] Schuchat (1999) Lancet 353(9146):51-6.
[112] WO02/34771.
[113] Dale (1999) Infect Dis Clin North Am 13:227-43, viii.
[114] Ferretti et al. (2001) PNAS USA 98: 4658-4663.
[115] Kuroda et al. (2001) Lancet 357(9264):1225-1240; see also pages 1218-1219.
[116] Anderson (2000) Vaccine 19 Suppl 1:S59-65.
[117] Kahn (2000) Curr Opin Pediatr 12:257-262.
[118] Crowe (1995) Vaccine 13:415-421.
[119] J Toxicol Clin Toxicol (2001) 39:85-100.
[120] Demicheli et al. (1998) Vaccine 16:880-884.
[121] Stepanov et al. (1996) J Biotechnol 44:155-160.
[122] EP-A-0139417.
[123] Harper et al. (2004) Lancet 364(9447):1757-65.
[124] Charalambous & Feavers (2001) J Med Microbiol 50:937-939.
[125] WO01/52885.
[126] Bjune et al. (1991) Lancet 338(8775):1093-1096.
[127] Fukasawa et al. (1999) Vaccine 17:2951-2958.
[128] Rosenqvist et al. (1998) Dev. Biol. Stand. 92:323-333.
[129] WO2004/032958.
[130] WO96/37624.
[131] WO98/46262.
[132] Huckriede et al. (2003) Methods Enzymol 373:74-91.
[133] Herlocher et al. (2004) J Infect Dis 190(9):1627-30.
[134] Le et al. (2005) Nature 437(7062):1108.
[135] Hoffmann et al. (2002) Vaccine 20:3165-3170.
[136] Subbarao et al. (2003) Virology 305:192-200.
[137] Liu et al. (2003) Virology 314:580-590.
[138] Ozaki et al. (2004) J. Virol. 78:1851-1857.
[139] Webby et al (2004)Lancet 363:1099-1103.
[140] WO00/60050.
[141] WO01/04333.
[142] U.S. Pat. No. 6,649,372.
[143] Neumann et al. (2005) Proc Natl Acad Sci USA 102:16825-9.
[144] WO01/83794.
[145] Hoffmann et al. (2000) Virology 267(2):310-7.
[146] WO97/37000.
[147] Brands et al. (1999) Dev Biol Stand 98:93-100.
[148] Halperin et al. (2002) Vaccine 20:1240-7.
[149] Tree et al. (2001) Vaccine 19:3444-50.
[150] Kistner et al. (1998) Vaccine 16:960-8.
[151] Kistner et al. (1999) Dev Biol Stand 98:101-110.
[152] Bruhl et al. (2000) Vaccine 19:1149-58.
[153] Pau et al. (2001) Vaccine 19:2716-21.
[154] http://www.atcc.org/
[155] http://locus.umdnj.edu/
[156] WO03/076601.
[157] WO2005/042728.
[158] WO03/043415.
[159] Lundblad (2001) Biotechnology and Applied Biochemistry 34:195-197.
[160] Guidance for Industry: Bioanalytical Method Validation. U.S. Department of Health and Human Services Food and Drug Administration Center for Drug Evaluation and Research (CDER) Center for Veterinary Medicine (CVM). May 2001.
[161] Ji et al. (2002) Biotechniques. 32:1162-7.
[162] Briggs (1991) J Parenter Sci Technol. 45:7-12.
[163] Lahijani et al. (1998)Hum Gene Ther. 9:1173-80.
[164] Lokteff et al. (2001) Biologicals. 29:123-32.
[165] EP-B-0870508.
[166] U.S. Pat. No. 5,948,410.
[167] WO97/37001.
[168] WO01/22992.
[169] Hehme et al. (2004) Virus Res. 103(1-2):163-71.
[170] Treanor et al. (1996) J Infect Dis 173:1467-70.
[171] Keitel et al. (1996) Clin Diagn Lab Immunol 3:507-10.
[172] WO00/56365.
[173] Gennaro (2000) Remington: The Science and Practice of Pharmacy. 20th ed ISBN: 0683306472
[174] U.S. Pat. No. 6,355,271.
[175] WO00/23105.
[176] WO01/22972.
[177] Kandimalla et al. (2003) Nucleic Acids Research 31:2393-2400.
[178] WO02/26757.
[179] WO99/62923.
[180] Krieg (2003) Nature Medicine 9:831-835.
[181] McCluskie et al. (2002) FEMS Immunology and Medical Microbiology 32:179-185.
[182] WO98/40100.
[183] U.S. Pat. No. 6,207,646.
[184] U.S. Pat. No. 6,239,116.
[185] U.S. Pat. No. 6,429,199.
[186] Kandimalla et al. (2003) Biochemical Society Transactions 31 (part 3):654-658.
[187] Blackwell et al. (2003) J Immunol 170:4061-4068.
[188] Krieg (2002) Trends Immunol 23:64-65.
[189] WO01/95935.
[190] Kandimalla et al. (2003) BBRC 306:948-953.
[191] Bhagat et al. (2003) BBRC 300:853-861.
[192] WO03/035836.
[193] Myers et al. (1990) pages 145-156 of Cellular and molecular aspects of endotoxin reactions.
[194] Ulrich (2000) Chapter 16 (pages 273-282) of reference 250.
[195] Johnson et al. (1999) J Med Chem 42:4640-9.
[196] Baldrick et al. (2002) Regulatory Toxicol Pharmacol 35:398-413.
[197] UK patent application GB-A-2220211.
[198] U.S. Pat. No. 4,680,338.
[199] U.S. Pat. No. 4,988,815.
[200] WO92/15582.
[201] Stanley (2002) Clin Exp Dermatol 27:571-577.
[202] Wu et al. (2004) Antiviral Res. 64(2):79-83.
[203] Vasilakos et al. (2000) Cell Immunol. 204(1):64-74.
[204] U.S. Pat. Nos. 4,689,338, 4,929,624, 5,238,944, 5,266,575, 5,268,376, 5,346,905, 5,352,784, 5,389,640, 5,395,937, 5,482,936, 5,494,916, 5,525,612, 6,083,505, 6,440,992, 6,627,640, 6,656,938, 6,660,735, 6,660,747, 6,664,260, 6,664,264, 6,664,265, 6,667,312, 6,670,372, 6,677,347, 6,677,348, 6,677,349, 6,683,088, 6,703,402, 6,743,920, 6,800,624, 6,809,203, 6,888,000 and 6,924,293.
[205] Jones (2003) Curr Opin Investig Drugs 4:214-218.
[206] WO2004/060308.
[207] WO2004/064759.
[208] U.S. Pat. No. 6,924,271.
[209] US2005/0070556.
[210] U.S. Pat. No. 5,658,731.
[211] U.S. Pat. No. 5,011,828.
[212] WO2004/87153.
[213] U.S. Pat. No. 6,605,617.
[214] WO02/18383.
[215] WO2004/018455.
[216] WO03/082272.
[217] WO2006/002422.
[218] Johnson et al. (1999) Bioorg Med Chem Lett 9:2273-2278.
[219] Evans et al. (2003) Expert Rev Vaccines 2:219-229.
[220] Andrianov et al. (1998) Biomaterials 19:109-115.
[221] Payne et al. (1998) Adv Drug Delivery Review 31:185-196.
[222] U.S. Pat. No. 5,057,540.
[223] WO96/33739.
[224] EP-A-0109942.
[225] WO96/11711.
[226] WO00/07621.
[227] Barr et al. (1998) Advanced Drug Delivery Reviews 32:247-271.
[228] Sjolanderet et al. (1998) Advanced Drug Delivery Reviews 32:321-338.
[229] Pizza et al (2000) Int J Med Microbiol 290:455-461.
[230] WO95/17211.
[231] WO98/42375.
[232] Singh et al (2001) J Cont Release 70:267-276.
[233] WO99/27960.
[234] U.S. Pat. No. 6,090,406
[235] U.S. Pat. No. 5,916,588
[236] EP-A-0626169.
[237] WO99/52549.
[238] WO01/21207.
[239] WO01/21152.
[240] Signorelli & Hadden (2003) Int Immunopharmacol 3(8):1177-86.
[241] WO2004/064715.
[242] Cooper (1995) Pharm Biotechnol 6:559-80.
[243] WO03/011223.
[244] Meraldi et al. (2003) Vaccine 21:2485-2491.
[245] Pajak et al. (2003) Vaccine 21:836-842.
[246] U.S. Pat. No. 6,586,409.
[247] Wong et al. (2003) J Clin Pharmacol 43(7):735-42.
[248] US2005/0215517.
[249] Vaccine Design: The Subunit and Adjuvant Approach (eds. Powell & Newman) Plenum Press 1995 (ISBN 0-306-44867-X).
[250] Vaccine Adjuvants: Preparation Methods and Research Protocols (Volume 42 of Methods in Molecular Medicine series). ISBN: 1-59259-083-7. Ed. O'Hagan.
[251] WO98/20734.
[252] WO01/30390
[253] Galli et al, PNAS, 2007, 104(1): 3984-3989
[254] Claassen E et al, Res Immunol., 1992, 143(5):478-83
[255] Vollmer J et al, Eur J Immunol., 2004, 34(1):251-62
[256] Lindblad, E. B., Immunol. Cell Biol., 2004, 82: 497-505
[257] Podda A, et al, Expert Rev Vaccines, 2003 2(2):197-203
[258] Bemasconi et al, Science, 2002, 298(5601):2199-202

1. A composition comprising: a) a CD1d ligand; and b) an antigen from group B streptococcus.

2. A composition comprising: a) a CD1d ligand; and b) an antigen from Neisseria meningitidis.

3. A composition comprising: a) a CD1d ligand; and b) an influenza virus antigen.

4-6. (canceled)

7. A method of raising an immune response in a patient, comprising administering to a patient a CD1d ligand and an antigen from group B streptococcus.

8. A method of raising an immune response in a patient, comprising administering to a patient CD1d ligand and an antigen from Neisseria meningitidis.

9. A method of raising an immune response in a patient, comprising administering to a patient a CD1d ligand and an influenza virus antigen.

10. The method of claim 7 wherein the antigen and CD1d ligand are administered simultaneously, sequentially or separately.

11. The method of claim 8 wherein the antigen and CD1d ligand are administered simultaneously, sequentially or separately.

12. The method of claim 9 wherein the antigen Cd1d ligand are administered simultaneously, sequentially or separately.

13-15. (canceled)

16. A method according to any one of claims 7 to 12 wherein the amount of CD1d ligand administered to said patient is less than 10 μg/kg patient bodyweight.

17. A kit comprising: (a) an antigen from group B streptococcus, an antigen from Neisseria meningitidis or an influenza virus antigen; and (b) a CD1d ligand.

18. A method of inducing long-term immunological memory to an antigen in a patient in need thereof comprising administering to said patient a composition comprising:
a) said antigen; and
b) a CD1d ligand,
such that the number and/or frequency of doses of said composition necessary for said patient to be capable of raising an immune response to subsequent exposure to said antigen is reduced compared to administration of said antigen in the absence of a CD1d ligand.

19. A method according to claim 18 wherein the number and/or frequency of doses of said composition necessary for said patient to be capable of raising a protective immune response to subsequent exposure to said antigen is reduced compared to administration of said antigen in the absence of a CD1d ligand.

20. A method according to claim 19 wherein the number of doses of said composition necessary for said patient to be capable of raising a protective immune response to subsequent exposure to said antigen is reduced compared to administration of said antigen in the absence of a CD1d ligand.

21. A method according to claim 20 wherein the number of doses required to induce a protective immune response is reduced to a single priming dose.

22. A method according claim 19 wherein the frequency of booster doses of said composition necessary for said patient to be capable of raising a protective immune response to subsequent exposure to said antigen is reduced compared to administration of said antigen in the absence of a CD1d ligand.

23. A method according to claim 22 wherein booster doses are administered at intervals of more than one year.

24. A method according to claim 23 wherein the requirement for booster doses is completely eliminated.

25. A method of inducing an immune response against an antigen in a patient comprising administering to said patient:
a) said antigen; and
b) a CD1d ligand,
wherein said antigen and a CD1d ligand were also administered to said patient more than one year previously.

26. (canceled)

27. A method according to claim 25 wherein the immune response is a protective immune response.

28. A method of claim 25 wherein the antigen and a CD1d ligand are administered simultaneously, sequentially or separately.

29. A method of claim 18 or claim 25 wherein the amount of CD1d ligand administered to said patient is less than 10 μg/kg patient bodyweight.

30. A method of inducing an immune response against an antigen in a patient comprising administering to said patient:
a) said antigen; and
b) a CD1d ligand,
wherein the amount of CD1d ligand included in the composition is less than 10 μg/kg patient bodyweight.

31. (canceled)

32. A method of claim 30 wherein the immune response is a protective immune response.

33. A method claim 30 wherein the CD1d ligand and antigen are administered simultaneously, sequentially or separately.

34. A method of any one of claims 18, 25 and 30, wherein the antigen is a saccharide antigen conjugated to a carrier protein.

35. A method of any one of claims 18, 25 and 30, wherein the antigen is a protein antigen.

36. A method, composition or kit according to any previous claim wherein the CD1d ligand activates invariant NKT cells.

37. A method, composition or kit according to any previous claim wherein the CD1d ligand increases the levels of IFN-γ, IL-4 and IL-13 secreted by invariant NKT cells compared to the levels of IFN-γ, IL-4 and IL-13 secreted by invariant NKT cells in the absence of the CD1d ligand.

38. A method, composition or kit according to any previous claim wherein the CD1d ligand is a glycolipid.

39. A method, composition or kit according to any previous claim wherein the CD1d ligand is an α-glycosylceramide.

40. A method, composition or kit according to any previous claim wherein the CD1d ligand is α-galactosylceramide or an analog thereof.

41. A method, composition or kit according to any previous claim wherein the CD1d ligand is an α-galactosylceramide analog selected from KRN7000, OCH or CRONY-101.
